(19) United States
(12) Patent Application Publication
(10) Pub. No.: US 2019/0192229 A1
(43) Pub. Date: Jun. 27, 2019

(54) SYSTEM AND METHOD FOR GUIDING INVASIVE MEDICAL TREATMENT PROCEDURES BASED UPON ENHANCED CONTRAST-MODE ULTRASOUND IMAGING

(71) Applicants: The Charles Stark Draper Laboratory, Inc., Cambridge, MA (US); The Board of Trustees of the Leland Stanford Junior Univesity, Stanford, CA (US)

(72) Inventors: Andrew A. Berlin, Lexington, MA (US); Juergen K. Willmann (Deceased), MD, Stanford, CA (US); Mon Y. Young, Qunicy, MA (US)

(21) Appl. No.: 16/198,702

(22) Filed: Nov. 21, 2018

Related U.S. Application Data
(63) Continuation-in-part of application No. 16/051,426, filed on Jul. 31, 2018.
(60) Provisional application No. 62/589,491, filed on Nov. 21, 2017.

(51) Int. Cl. A61B 34/20; A61B 8/08; A61B 5/055; A61B 6/03; A61B 8/06

(52) U.S. Cl. CPC A61B 34/20; A61B 8/481; A61B 8/0841; A61B 8/5246; A61B 8/5261; A61B 8/488; A61B 2505/05; A61B 6/032; A61B 8/06; A61B 8/5269; A61B 2034/2051; A61B 2034/2063; A61B 5/055

(57) ABSTRACT

This invention provides a system and method for guiding a surgical instrument based on information obtained using enhanced contrast-mode ultrasound. The enhanced information can be added to information or images obtained in one or more imaging modes. Various pieces of information can be combined and composited, including information regarding tumors, blood vessels, location information, confidence levels, and other information, and can be composited into operative imaging.

### RELATED APPLICATION

This application claims the benefit of co-pending U.S. Provisional Application Ser. No. 62/589,491, entitled SYSTEM AND METHOD FOR DYNAMIC BACKGROUND SIGNAL REMOVAL AND RESOLVING REGIONS OF INTEREST IN CONTRAST-ENHANCED ULTRASOUND IMAGES, filed Nov. 21, 2017, the teachings of which are expressly incorporated herein by reference.

### FIELD OF THE INVENTION

This invention relates to medical imaging and more particularly to processing and analysis of contrast-enhanced ultrasound images

### BACKGROUND OF THE INVENTION

Ultrasound is sound waves with frequencies which are significantly higher than those audible to humans (>20,000 Hz). Ultrasonic images also known as sonograms are made by sending pulses of ultrasound into tissue using a transducer (also termed a probe). The sound echoes off the tissue, with different tissues reflecting varying degrees of sound. These echoes are recorded and displayed as an image to the operator.

A recent development in medical ultrasound imaging technology is known as ultrasound contrast imaging. This mode of medical ultrasound imaging employs microbubbles as a contrast enhancing agent (also termed a “contrast agent”) that may or may not be molecularly targeted. Microbubble-based contrast media is administrated intravenously into a patient's blood stream during the medical ultrasonography examination. The microbubbles being too large in diameter, they stay confined in blood vessels and cannot extravasate towards the interstitial fluid. An ultrasound contrast media is therefore purely intravascular, making it an ideal agent to image organ microvascularization for diagnostic purposes. A typical clinical use of contrast ultrasonography is detection of a hypervascular metastatic tumor, which exhibits a contrast uptake (kinetics of microbubbles concentration in blood circulation) faster than healthy biological tissue surrounding the tumor. Other clinical applications using contrast exist, such as in echocardiography to improve delineation of left ventricle for visually checking contractibility of heart after a myocardial infarction.

More generally, microbubbles have great potential to make it easier to detect disease early, to monitor disease progression and drug effectiveness, and to guide surgical procedures such as biopsies. However, existing approaches to detection of the accumulation of targeted contrast agents in living tissue using ultrasound are not sufficient to achieve this potential. Existing techniques are either not suitable for widespread use in humans, due to techniques that necessitate microbubble destruction using a high burst of acoustic energy that risks damage to blood vessels, or due to measurement challenges in which ultrasound signals from other sources are confounded with the signal from the accumulating microbubbles, leading to low-confidence measurements. Thus, existing techniques are generally incompatible with the real-world constraints (energy levels, duration of exam, geometries involved, etc.) of imaging in humans, and lack signal clarity required to reliably disambiguate contrast agent from other sources of signal intensity. More particularly, it can be challenging to distinguish chemically bound contrast agent versus unbound contrast agent, the latter of which can vary greatly in accumulation and flow between acquired time-based image frames. This leads to full or partial occlusion of features of interest, such as tumorous tissue, as well as to camouflaging of the bound contrast agent by unbound contrast agent or background signal.

With the increasing prevalence of robot-guided procedures, it is also increasingly desirable to provide the robotic system with accurate data that can be employed in directing surgical instruments to, and around, an internal region of the body. Likewise, manual procedures can benefit from more accurate, real-time images of the internal region.

### SUMMARY OF THE INVENTION

This invention overcomes disadvantages of the prior art by providing a system and method for removal of various features from images acquired by an ultrasound scanner in the presence of molecularly bound contrast agent. The removed features can include background features, camouflaging features, confounding artifacts, and/or other features. The system and method employs novel techniques that are compatible with the real-world constraints (i.e. energy levels, duration of exam, geometries involved, etc.) of imaging in mammalian tissue (e.g. human organ tissues containing lesions/tumors), while providing the dramatically improved signal clarity required to reliably disambiguate contrast agent from other sources of signal intensity.

In an embodiment, the system and method operates to effectively quantitate molecularly bound contrast agent, performing a number of advantageous actions including, but not limited to (1) disambiguating signal intensity due to molecularly bound contrast agent from signal that is due to freely-flowing contrast agent; (2) disambiguating signal due to molecularly bound contrast agent from signal due to non-specifically immobilized contrast agent, i.e. contrast agent that is stationary, but that has accreted in the tissue region, or occupies a fixed location, and is not otherwise part of a molecular binding-induced accumulation of contrast agent over time; (3) disambiguating signal intensity that is due to molecularly bound contrast agent from signal associated with imaging artifacts such as echoes, reflections, and resonances; (4) disambiguating signal due to molecularly bound contrast agent from tissue signal that has not been adequately suppressed. For example, certain types of connective tissue generate sustained signals containing harmonics that very closely resemble those produced by contrast agents such as microbubbles, and hence, are not suppressed by the existing generation of contrast agent-selective filters used in ultrasound imaging machines. In other words, sustained signal from tissue that is present in the contrast-mode image even before the contrast agent has been administered; and (5) disambiguating signal that is due to molecularly bound contrast agent from intermittent signals that arise due to tissue, which sometimes elude the contrast-mode filters to create short, localized bursts of intensity in the contrast mode image.

The illustrative system and method also provides novel arrangements that permit the accumulation of contrast agent due to molecular binding to be more clearly quantified and disambiguated from other sources of ultrasound image intensity. These arrangements include (1) an overall system architecture, for computationally-enhanced ultrasound imaging of contrast agent accumulation that combines windowing and flow dynamics modeling approaches to provide detection of contrast agent accumulation with far greater confidence than is achieved by existing approaches. i.e. fewer false positive and false negative results; (2) novel methods for background model generation that account for not only sustained signal sources, but also the bursty signal sources associated with insufficiently suppressed signals; (3) occlusion identification and compensation modules, including identification of a previously unrecognized effect in which background signal is sometimes added to signal from contrast agent, and sometimes occluded by contrast agent, and development of detection and compensation mechanisms to exploit this occlusion effect; (4) measurement window image fusion processes, which provide robust multi-frame image fusion to form statistical windows over time intervals, with novel models and methods for estimating contrast agent concentration within each measurement window; (5) multi-window refinement processes to refine contrast accumulation estimates based on analysis and model/expectation-fitting to windowed data rather than to raw signal intensity information; (6) region of interest segmentation processes for automatic segmentation of an image to identify regions of interest that share similar contrast agent accumulation characteristics; and (7) result presentation tools that generate a user-friendly representation of concentration estimates and confidence metrics, enabling end users to observe not only where high concentration of contrast agent is estimated, but also regions where low concentration is estimated and regions where concentration is uncertain. Use of this information can provide real-time feedback during the ultrasound examination, suitable for use in manual or automatic adjustment of imaging parameters, such as probe position, energy levels, and sample rate.

In an illustrative embodiment, a method for performing a surgical procedure can include administering an ultrasound contrast agent to a patient, performing ultrasound imaging of at least a portion of the patient using contrast-mode ultrasound to obtain at least one ultrasound image of the patient, performing statistical analysis of the at least one contrast-mode ultrasound image to enhance the clarity of the at least one contrast-mode ultrasound image, and guiding a surgical tool using information gained from the at least one enhanced contrast mode ultrasound image. Performing statistical analysis of the at least one ultrasound image can include performing statistical analysis of multiple-image windows to identify pixel intensity caused by molecularly bound contrast agent. Performing ultrasound imaging of a portion of the patient using contrast-mode ultrasound to obtain at least one ultrasound image of the patient can include performing operative imaging of the patient to obtain operative images of the patient using contrast-mode ultrasound, and wherein guiding a surgical tool using information gained from the at least one enhanced contrast mode ultrasound image can include guiding the surgical tool using the enhanced operative images. The method can include performing operative imaging of the patient to obtain operative images using B-mode ultrasound, Doppler-mode ultrasound, Magnetic Resonance Imaging (MRI), CT scan, or fluoroscopy, and guiding a surgical tool using information gained from the at least one enhanced contrast mode ultrasound image can include adding the information gained from the contrast-mode ultrasound to the operative images. Adding the information gained from the contrast-mode ultrasound to the operative imaging can include annotating the operative images to include regions of interest determined from the statistical analysis of pixel intensity in the contrast-mode ultrasound. The method can include tracking features using B-mode feature tracking relative to key landmarks, and updating locations of regions of interest relative to tracked features. Adding the information gained from the contrast-mode ultrasound to the operative imaging can include annotating the operative images to include biological margins determined from the statistical analysis of pixel intensity in the contrast-mode ultrasound. The method can include performing operative imaging of the patient to obtain operative images, and guiding a surgical tool using information gained from the at least one enhanced contrast mode ultrasound image can include annotating the operative images to include regions of interest determined from the statistical analysis of pixel intensity in the contrast-mode ultrasound, the annotating being relative to a landmark. The method can include performing operative imaging of the patient to obtain operative images, and wherein guiding a surgical tool using information gained from the at least one enhanced contrast mode ultrasound image further comprises annotating the operative images to include biological margins determined from the statistical analysis of pixel intensity in the contrast-mode ultrasound, the annotating relative to a landmark. The method can include adding a color-coding to the one or more ultrasound images to indicate confidence levels in different portions of the one or more ultrasound images. The method can include adding a color-coding to the one or more ultrasound images to indicate confidence levels in different portions of the one or more ultrasound images. The method can include indicating to a user that a predetermined confidence requirement for confidence in the enhanced ultrasound image has been met so that surgery can proceed with confidence.

In an illustrative embodiment, a method for performing a surgical procedure can include administering at least one ultrasound contrast agent to a patient, performing at least one ultrasound imaging of at least a portion of the patient using contrast-mode ultrasound to obtain at least one ultrasound image of the patient, measuring tissue perfusion in at least one of the at least one ultrasound images of the patient to obtain tissue perfusion information indicating areas of blood supply flow, identifying locations of bound contrast agent in at least one of the at least one ultrasound images of the patient to obtain bound contrast location information, integrating the tissue perfusion information and the bound contrast location information into a correlated information set that describes perfusion information and bound contrast location information simultaneously, and guiding a surgical tool using the correlated information set. Integrating the tissue perfusion information and the bound contrast location information into a correlated information set can include integrating the tissue perfusion information and the bound contrast location information into an integrated image. Administering at least one ultrasound contrast agent to a patient can include administering a molecularly targeted ultrasound agent, and identifying locations of bound contrast agent in at least one of the at least one ultrasound images of the patent can include annotating a region of interest indicated by molecularly bound contrast agent. Guiding a surgical tool using the correlated information set can include guiding a surgical instrument to the region of interest, and can include avoiding areas of blood supply flow. Guiding a surgical tool using the correlated information set can include guiding a surgical instrument to remove the region of interest, and can include disrupting blood supply flow to the region of interest. Administering at least one ultrasound contrast agent to a patient can include administering a non-molecularly targeted ultrasound contrast agent, and measuring tissue perfusion in at least one of the at least one ultrasound images of the patient to obtain tissue perfusion information indicating areas of blood supply flow can include analyzing flow patterns of the non-molecularly targeted ultrasound contrast agent. Measuring tissue perfusion in at least one of the at least one ultrasound images of the patient to obtain tissue perfusion information indicating areas of blood supply flow can include performing statistical analysis to analyze flow patterns of the molecularly targeted ultrasound contrast agent. Performing statistical analysis can include performing a maximum intensity projection to approximate blood flow patterns. Performing statistical analysis can include determining a difference between the maximum intensity projection and a bound contrast agent projection, and the bound contrast agent projection can be acquired via minimum intensity projection or statistical estimation. Annotating a region of interest indicated by molecularly bound contrast agent can include defining a biological margin using statistical analysis. Performing statistical analysis can include performing a percentage intensity projection. Performing a percentage intensity projection can include performing a percentage intensity projection in a range of approximately 70% to approximately 90%. The method can include subtracting a minimum intensity projection from the percentage intensity projection, the minimum intensity projection in a range of approximately 1% to approximately 10%.

### BRIEF DESCRIPTION OF THE DRAWINGS

The invention description below refers to the accompanying drawings, of which:

FIG. 1 is diagram of a generalized ultrasound scanning system including an interconnected processing device (e.g. a PC) that implements the systems and methods in accordance with illustrative embodiments;

FIG. 2 is a flow diagram of an overall procedure for acquiring, processing and transmission of image data in a contrast-mode-based ultrasound scanning environment, including background removal in accordance with embodiments herein;

FIG. 3 is a diagram showing the acquisition and processing of frames into overlapping, composite windows over time;

FIG. 4 is a schematic representation of image data of a scanned tissue site (e.g. tissue with a pathology such as, by way of non-limiting example, a cancer lesion/tumor), based upon a brightness-mode ultrasound image acquired prior to contrast agent administration;

FIG. 5 is a schematic representation of image data from the scanned tissue site of FIG. 4, showing a contrast-mode view, prior to administration of contrast agent;

FIG. 6 is a schematic representation of image data from the scanned tissue site of FIG. 4, showing a composite image formed by fusion of (e.g.) sixteen (16) contrast-mode frames via a Maximum Intensity Projection, prior to contrast agent administration;

FIG. 7 is a flow diagram showing a procedure for generating a background (or signal) model for use in the background removal procedure of FIG. 2, where pre-contrast-agent-arrival image data/examples is/are not generally unavailable;

FIG. 8 is a schematic representation of image data from the scanned tissue site of FIG. 4, showing an estimate of contrast agent accumulation captured via imaging of a tumor, captured approximately (e.g.) five minutes after administration of contrast agent;

FIG. 9 is a schematic representation of image data from the scanned tissue site of FIG. 4, showing an estimate of contrast agent accumulation after statistics based background removal (no use of pre-arrival examples) and morphological closure, by way of comparison with FIG. 8;

FIG. 10 is a schematic representation of image data from the scanned tissue site of FIG. 4, showing an estimate of bound contrast agent concentration, after further refinement based on the use of brightness-mode intensity information;

FIG. 11 is a schematic representation of image data from the scanned tissue site of FIG. 4, showing an estimate of high-spatial-resolution concentration derived from image data prior to background removal/elimination in accordance with an embodiment, masked by areas in which the background-subtracted signal strength is greater than a threshold (in this example the threshold is set to 0);

FIG. 12 is a more detailed schematic representation of image data based upon the scanned tissue site of FIG. 4, again showing a version of a brightness-mode-based ultrasound image data, prior to contrast agent administration;

FIG. 13 is a schematic representation of image data from the scanned tissue site of FIG. 12, from the same vantage point/perspective, showing a contrast-mode-based ultrasound image, acquired a few seconds subsequent to the image of FIG. 12, and also prior to contrast agent administration;

FIG. 14 is a schematic representation of image data from the scanned tissue site of FIG. 12, from the same vantage point/perspective, showing a contrast-mode-based ultrasound image, acquired after arrival of targeted contrast agent, as such targeted contrast agent accumulates, and as circulating contrast agent perfuses the tissue;

FIG. 15 is a schematic representation of image data based upon the scanned tissue site of FIG. 12, showing an image generated using a minimum-intensity projection filter over a window of (e.g.) 20-frames at (e.g.) 1 frame-per-second, exhibiting an enhanced signal prior to contrast-agent administration;

FIG. 16 is a schematic representation of image data from the scanned tissue site of FIG. 15, showing an image exhibiting an enhanced signal following contrast-agent administration, and by way of comparison with FIG. 15, showing that arrival of the contrast agent decreases the enhanced signal intensity in the region of the tumor by (e.g.) 43%;

FIG. 17A is a schematic representation of image data from a region of interest in the scanned tissue site described above, showing the first step in constructing a model of the background (or signal) from multiple frames prior to contrast agent arrival;

FIG. 17B is a schematic representation of image data from a region of interest in FIG. 17A, showing the next step in constructing a model of the background (or signal) from multiple frames, in which contrast agent arrival has occurred, but prior to background removal;

FIG. 17C is a schematic representation of image data from a region of interest in FIG. 17A, showing the next step in constructing a model of the background (or signal) from multiple frames after contrast agent arrival, and after undergoing subtractive background removal, in which a black hole characteristic is displayed;

FIG. 17D is a schematic representation of image data from a region of interest in FIG. 17A, showing the next step in constructing a model of the background (or signal) from multiple frames, following occlusion-compensated background removal in which the black hole is filled with image data;

FIG. 18 is a diagram of a table showing a measurement windowing approach, grouping adjacent samples to form statistical measurement windows; for use in a measurement window image fusion step according to embodiments of the system and method herein;

FIG. 19 is a representative graph showing an exemplary set of raw ultrasound data showing the curve of a contrast agent signal in pathologies, for example relative to a cancer lesion, compared with the curve of a signal that is, by way of example, normal (non-cancerous) tissue;

FIG. 20 is a representative, exemplary graph showing estimates of intensity in each measurement window due to bound (stationary) contrast agent for normal and diseased tissue, for example cancerous tissue, using (for example) alpha (α)=2.0.

FIG. 21 is a representative, exemplary graph showing more conservative estimates of intensity in each measurement window that are due to bound (stationary) contrast agent for normal and diseased tissue, for example a cancerous tissue, using (for example) alpha (α)=2.5;

FIG. 22 is a representation of an exemplary segmented image representation showing regions where the estimated bound contrast agent accumulation exceeds a threshold T computed based on an estimated bound contrast agent intensity value at least K=3 standard deviations above the mean intensity of the image;

FIG. 23 is a representation of an exemplary segmented image showing regions at initial detection for an exemplary measurement window of size 15 that extends to a time point (e.g.) 15 seconds beyond contrast agent arrival;

FIG. 24 is a representation of an exemplary segmented image showing detection results for a measurement window of exemplary measurement window size 15 that extends to a time point (e.g.) 38 seconds beyond contrast agent arrival; FIGS. 25A and 25B are, respectively, schematic image representations of detection results overlaid onto raw image data for the measurement window that ends (e.g.) 15 seconds after contrast agent arrival, and for the measurement window that ends (e.g.) 19 seconds after contrast agent arrival;

FIG. 26 is a diagram showing an arrangement for performing robotic (or manually) guided invasive procedures using enhanced ultrasound imagery in accordance with the systems and methods herein; and

FIG. 27 is a generalized flow diagram of a procedure for performing procedures in accordance with the arrangement of FIG. 26.

### DETAILED DESCRIPTION

I. System Overview

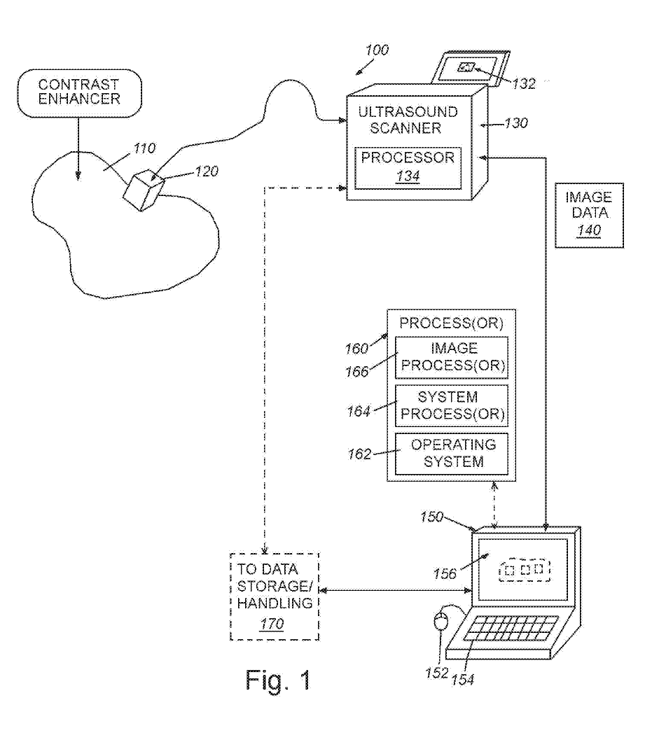

FIG. 1 shows a diagram of a generalized system 100 for scanning tissue 110 (e.g. human or mammalian) using ultrasound energy. The exemplary system 100 includes a transducer/probe 120, which is shown held against the tissue in an appropriate orientation using freehand guidance or a mechanical device (e.g. a robotic manipulator, such as the da Vinci® surgical robot, available from Intuitive Surgical, Inc. of Sunnyvale, Calif.). The probe 120 defines a transceiver that transmits ultrasound energy to the tissue, and receives echoes/reflections that are converted into electromagnetic signals. These signals are received by the base scanner unit 130, which can be any acceptable manufacturer and model—for example, Philips, Siemens, HP, General Electric, etc. The exemplary base scanner unit 130 includes an onboard display 132 that allows for local viewing and control of images acquired by the probe. It can include touch screen functions to allow a user to interface with the base unit 130. Alternatively, control can be provided by an alternate user interface implementation (e.g. keyboard, trackball/touchpad, buttons, etc.). The acquired image data is manipulated by the processor 134 and associated image processing software/hardware. Image data 140 can also be transmitted to a PC, server or other processing device (including the scanner's internal processor) 150. The processing device 150 includes a user interface (e.g. mouse 152, keyboard 154, touchscreen 156, and the like). By way of non-limiting example, the device's process(or) includes an operating system 162, and various generic and custom system processes 164 (e.g. image manipulation software, analysis programs, such as MATLAB®, available from The Mathworks, Inc. of Natick, Mass.). The processing device's operational process(or) 160 can also include image processing software/hardware, including various processors/es (also termed “modules”) 166 for implementing the teachings of the illustrative embodiments herein.

The processing device 134 and/or scanning base unit 130 can be operatively connected with a data storage system (e.g. disks, solid state drives, network attached storage (NAS), storage area network (SAN), cloud-based storage, etc.) 170 that allows image data to be written to or read from the storage media. The stored image data can be retrieved to allow processing using the illustrative procedures herein and/or after such processing, by downstream processes. Image data can be stored in accordance with various formats including the well-known DICOM standard.

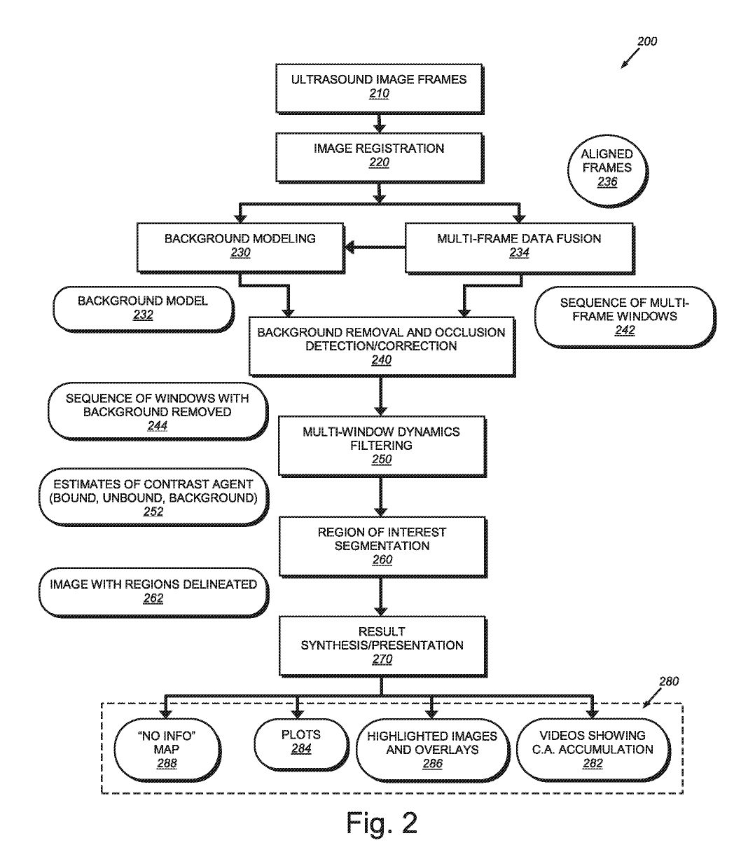

The illustrative embodiments teach novel techniques for quantitation of molecularly bound contrast agent in ultrasound imaging. These techniques are intended to integrate with an overall scanning/processing system architecture. FIG. 2 shows a procedure 200, within that architecture, which combines individual measurement approaches into a data flow pattern that enables substantially improved ability to detect and quantify contrast agent presence and dynamics. The illustrative system architecture and procedure 200 provides a combination of image fusion to form composite measurement windows, background (or signal) modeling and removal, and dynamic multi-window filtering to distinguish signal arising due to bound or accumulating contrast agent from other sources of image intensity.

In accordance with this overview, each step of the procedure is described herein briefly by way of basic understanding of the concepts presented herein. A more detailed explanation of the various steps follows in subsequent sections below.

As shown in the procedure 200, in block 210 ultrasound image frames are delivered from the scanner or another modality—for example a data store (e.g. 170 above) associated with the scanner base unit 130, or another processor 160—to the image processing module 166. The image frames received are typically registered with one another (aligned) in multiple dimensions in step 220. In the case of probe motion, such as a translation in one dimension in the plane of imaging, it is often possible to align frames by simple translation or via a deformable registration process (e.g. affine transformation). However, in general, as organs move and deform in the presence of breathing, blood flow, probe motion in the out-of-plane direction, etc., it is not practical to perfectly align multiple images taken at different points in time as 3D voxels translate in multiple degrees of freedom. Thus, it is desirable that the processes used to identify contrast agent accumulation be robust to imperfect frame-to-frame alignment. Note, that the use of more-advanced registration tools that account for deformation is expressly contemplated in further embodiments. More particularly, note that in implementations for which contemporaneously acquired contrast-mode and other ultrasound imaging modalities such as B-mode (brightness mode) images are available, it can be desirable to utilize motion/deformation features that are made evident in these alternative image sources, in combination with the contrast-mode data, to improve the image registration process, as is taught in Quantification of Bound Microbubbles in Ultrasound Molecular Imaging, Vierya Daeichin, Zeynettin Akkus, Ilya Skachkov, Klazina Kooiman, Andrew Needles, Judith Sluimer, Ben Janssen, Mat J. A. P. Daemen, Antonius F. W. van der Steen, Nico de Jong, and Johan G. Bosch, IEEE Transactions on Ultrasonics, Ferroelectrics, and Frequency Control, Vol. 62, No. 6, June 2015; which is incorporated herein by reference as useful background information. For detection of contrast agent, the Daeichin approach utilizes a so-called minimization approach, which is further described generally in EP Published Patent No. EP 1 951 124 B1, and related applications, entitled DETECTION OF IMMOBILIZED CONTRAST AGENT IN MEDICAL IMAGING APPLICATIONS BASED ON FLOW DYNAMICS ANALYSIS, filed Nov. 9, 2006, and published for grant Jan. 4, 2017.

In step 230, the procedure 200 performs background (or signal) modeling. This process module constructs a model 232 of the background signal, i.e. signal that is present in the contrast-mode images but that is not due to presence of the contrast agent. In an embodiment, this is performed by analyzing images acquired prior to arrival of contrast agent, for which all signal is characterized as background, since the contrast agent has not yet been introduced. In another embodiment, the background signal is estimated from images acquired after contrast agent arrival, based on certain differentiating statistical properties of the background signals.

In step 240, background removal and occlusion compensation occurs. The estimated background signal from step 230 (in accordance with any embodiment) is removed from the overall signal. Through techniques known in the literature (such as background removal, which is also termed “subtraction”) or optionally through the use of novel techniques described below, which account for the possibility that background has been occluded, i.e. is no longer present.

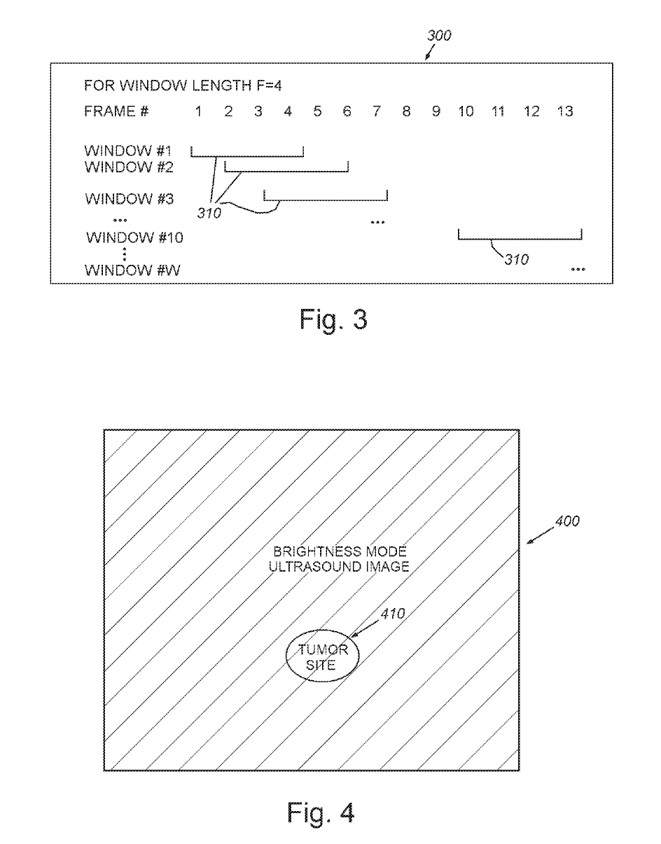

In performing background removal and occlusion detection in accordance with step 240, the procedure 200 also implements data fusion to form a sequence of measurement windows (242) in step 234. Each measurement window includes a variety of image and statistical data that collectively characterize the underlying information. As shown in the diagram 300 of FIG. 3, individual image frames 236 are composited to form a set of overlapping (represented by brackets 310), advancing, multi-frame measurement windows 242, as illustrated in the diagram 300 shown in FIG. 3. Each window W (e.g. Window #1-Window #10, etc.) comprises a set of samples acquired over a given time interval, which is represented by a number of frames (F) (acquired over a time period). In this case four (F=4) frames are composited into a window. Each window can define multiple properties, representing a composite behavior of the frames—for example, the MEAN value of the samples in a given window, the associated minimum, the maximum, the standard deviation, the Gaussian-weighted summation, and/or other statistical measures. In performing the above-described background removal, the overall signal is ideally suppressed in the contrast view mode of the scanner—allowing only the enhanced areas to appear visible. This generates a sequence of windows with background removed 244. In an embodiment, the statistics associated with each window are calculated (or updated) following background removal.

Each pixel within a measurement window has multiple time points (based on the frames F) associated with it. According to the procedure, for each pixel, and also for aggregations of pixels, statistical measurements are computed that represent the composite behavior of that pixel or region over the time frame represented by the window. For example, for each pixel, the MEAN value of the intensity present at that pixel location for all samples in the window, as well as the minimum intensity, maximum, standard deviation, frequency spectral properties, Gaussian-weighted summation of the pixel intensity with its spatial neighbors, and other statistical measures may be computed, on a window-by-window basis.

Once the frames have been composited into a sequence of windows, contrast agent concentration within each window is estimated as part of step 240. This can be achieved using the Minimum Intensity Projection (MIP) or Percent-Intensity-Projection approaches (PIP) (as taught the above-referenced IEEE publication). The MIP approach taught by Daeichin uses the lowest pixel intensity for each pixel across a measurement window as the value for that pixel. The PIP approach analyzes the pixel intensities for each pixel across a measurement window, and then identifies a pixel intensity value such as, for example, the pixel intensity value that is at the 20^{th} percentile out of the intensity values for that pixel, and then uses that 20^{th} percentile intensity value as the value for that pixel. Various embodiments can use pixel intensity values from approximately the 10^{th} percentile to approximately the 30^{th} percentile. Alternatively, one of the novel statistical-modeling approaches provided herein below can be employed.

The sequence of multi-frame windows with background removed 244 are passed to step 250, which provides multi-window dynamic filtering (refinement).

In certain imaging implementations, such as monitoring of arrival of a bolus of contrast agent and the initial binding dynamics of contrast particles to tissue of interest, particular dynamic behavior of the accumulation of contrast agent can be expected. For example, it is recognized that the concentration of contrast agent at a location where binding is occurring starts low and increases over time. By applying such types of expectations as filters, the system and method can reduce extraneous signals, and can disambiguate between stationary contrast agent that is accumulating due to targeted molecular binding (wherein a gradual increase in image intensity is expected) vs. stationary contrast agent that has become stationary simply because it has become stuck (wherein a one-time increase in image intensity is expected) due to a circulatory feature or the occasional non-specific binding event.

By applying dynamic accumulation models across measurement windows, utilizing the window-based statistical estimates of stationary contrast agent concentration, significantly improved results are obtained relative to current approaches, which attempt to fit accumulation models to the raw frame data directly. See Quantification of the binding kinetics of targeted ultrasound contrast agent for molecular imaging of cancer angiogenesis, by Simona Turco, Peter J. A. Frinking, Hessel Wijkstra, and Massimo Mischi, IEEE International Ultrasonics Symposium Proceedings, 2015, and Quantitative ultrasound molecular imaging by modeling the binding kinetics of targeted contrast agent, by Simona Turco, Isabelle Tardy, Peter Frinking, Hessel Wijkstra, and Massimo Mischi, Phys. Med. Biol. 62 (2017) 2449, which are incorporated herein by reference by way of useful background information.

The filtering/refinement step 250 yields estimates of contrast agent concentration, both bound and unbound, and background information 252. This is provided to the region of interest segmentation step 260. The refined concentration estimates are segmented by this step into regions having similar absolute intensity and/or dynamic intensity characteristics. These regions are then identified graphically (delineated) and are made available to be used as input to presentation tools.

The image(s) with concentration estimates (252) and/or delineated regions 262 generated in step 260 are presented to the result synthesis and presentation step 270. A variety of results 280 can be generated in user-presentable formats via an appropriate graphical user interface (or other media, such as print) with a user device (e.g. PC, smartphone, etc.). These include videos 282 showing accumulation of contrast agents over time (based on window-by-window concentration estimates), plots 284 showing properties of each measurement window, such as estimated contrast agent concentration over time, highlighted images and overlays showing accumulation locations, highlighted images and overlays 286 showing zero-accumulation locations (normal tissue), and highlighted images and overlays 288 that explicitly delineate areas where no reliable estimate regarding contrast agent concentration can be reached (for example, due to interference based upon imaging artifacts).

II. Detailed System and Method

Having described above the general system architecture and associated operational procedures, the following is a more detailed description of system components/modules and the various process steps associated with their operation.

A. Background/Signal Modeling

To differentiate signal derived from contrast agent from signal derived from other sources, a model is constructed of the background signal that is present in the acquired images, but that is not the result of contrast agent presence. Traditionally this is achieved via a frame-to-frame comparison between a frame taken just before contrast arrival, and a frame taken after contrast arrival. It is recognized that, in practical imaging conditions, short bursts of insufficiently suppressed background signal arising from tissue can be quite significant, as are variations in intensity triggered by occasional motion of a neighboring intense area of background triggered by probe-based and/or patient-based motion. In some imaging conditions, these intermittent variations in background intensity can account for as much as 33% of the signal variance. These bursts are present in addition to the sustained background signal that is commonly identified by methods in the literature. Note that the term “confounding signals” can include background signals such as various tissue leakage signals, imaging artifacts such as resonance, and bursting increases in background signal intensity, along with flowing contrast agent signal.

In conventional imaging applications, this intermittent bursting in background signal intensity is easily overlooked, as it only occurs at a few pixels at any given moment. However, in the case of quantification of bound contrast agent versus flowing contrast agent, for which a differentiating factor is intensity variation over time, these intermittent signals become significant. This variation due to bursting background intensity is blended with the variation that is associated with freely-flowing contrast agent shortly after injection, and in combination the confounding signals may be of comparable intensity to the signal arising from accumulating contrast agent. So elimination of this mostly-suppressed but occasionally intense bursting background signal is desirable if accurate diagnostic results are to be obtained from monitoring of molecular binding. Thus, this description provides various illustrative techniques/methods for constructing a model of the unsuppressed background signal that is generally present in contrast-mode ultrasound imaging, but that is not associated with contrast agent presence.

A first illustrative technique/method includes construction of a conservative background intensity model based on analysis of multiple images acquired prior to arrival of the contrast agent at the site of interest. A second illustrative technique/method involves use of statistical analysis applied to images acquired after arrival of contrast agent at the imaging site—for example, 5 minutes after introduction/injection of contrast agent. At this post-introduction time, there exists signal from bound contrast agent, background signal, and freely-flowing contrast agent present in the acquired images. Notably, as freely-flowing contrast agent circulates throughout the tissue, certain statistical properties of the intensity associated with unsuppressed background interact with the signals from the flow in a way that permits differentiation of locations where intensity is bright due to background from locations where intensity is bright due to flowing contrast agent. This permits an estimate of background signal intensity to be developed even without availability of a set of images acquired prior to contrast agent administration.

(i). Construction of Background Signal Model Using Pre-Contrast Arrival Images

Conventional ultrasound imaging is based on tissue reflections, and is referred to as brightness mode (or B-mode). FIG. 4 illustrates a schematic representation of an exemplary brightness-mode ultrasound image of tissue that contains a hidden pathology, for example a cancer lesion, that is generated as a result of one of the plurality of steps in the various processes described herein. Note, for purpose of this description, the actual image data is substituted for generalized textual descriptions and cross-hatching which is meant to represent the image generated by a particular, described step of the processes. In the representation of FIG. 4 (and other image representations herein), which can be an image of an organ or other appropriate site, the hatched/lined areas (typically displayed in an actual image as light areas) can broadly represent the signal received from tissue before infusion of contrast agent. This depicted representation of a brightness-mode ultrasound image includes a pathological lesion, for example, a cancer lesion/tumor (e.g. region 410) prior to contrast agent administration. All signal present is from tissue and would ideally be suppressed in the contrast-mode view to render contrast agent accumulation in the lesion more prominent for a practitioner to reliably identify. In various views herein the degree of brightness of the region is depicted by either single hatch marks (moderately bright) and (cross-hatched marks (significantly bright).

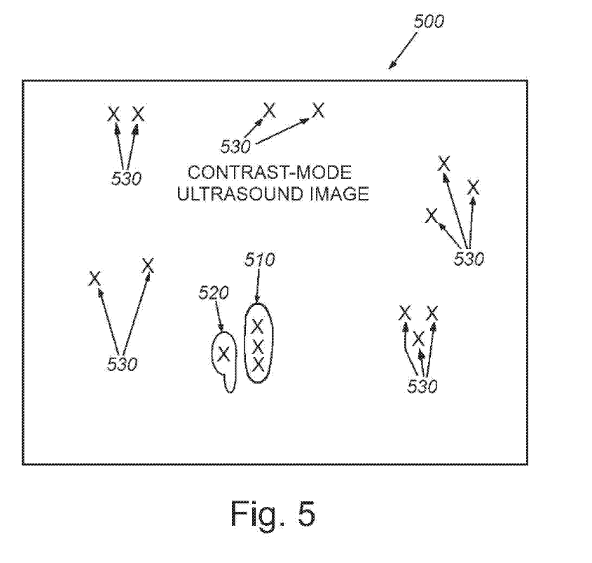

A variety of existing techniques exist to suppress this signal, such as the use of various harmonic frequency properties of microbubble contrast agent particles that differ from the properties of ordinary tissue background. A particularly popular technique, which is now typically incorporated into commercially available ultrasound equipment, is the contrast mode. Ideally, in contrast mode, all of the B-mode signal present would be suppressed, leaving only the signal from contrast agent. In practice, however, in real-world imaging, some of this signal may not be completely suppressed, causing image features to appear in contrast-mode even though the contrast agent has not yet been administered, as illustrated in the graphical representation of an exemplary schematic diagram of an image 500 in FIG. 5. The contrast-mode ultrasound image is acquired from the same pathology, for example, a cancer lesion from the same vantage point as that of the B-mode representation 400 of FIG. 4, also prior to contrast agent administration (possibly a few seconds later). Most of the signal from the tissue is suppressed by operation of contrast mode, yet in some locations, significant amounts of signal remain. In this case, one of the bright spots of remaining signal 520 is co-located with the pathology, for example, a cancer lesion/tumor region 510. In both intensity and texture, this tissue background signal often bears significant resemblance to the signal that results from the accumulation of targeted contrast agent, even though the contrast agent has not yet been administered. This type of artifact has significant potential to lead to false-positive diagnostic results, when it is misinterpreted as contrast agent. It can also lead to false-negative results when contrast agent in that location is erroneously interpreted by the practitioner as originating from tissue background. This background signal typically includes a few bright spots that are sustained over time, caused by imaging effects such as tissue leakage or tissue resonance effects. There are also many other spots (e.g. spots 530), whose location and intensity tends to vary over time, in an intermittent, or bursty, manner. While in any given frame, these spots are not significant, when intermingled (added to) signal from contrast agents, they can become a source of signal variation that is quite significant, often representing as much as 33% of the overall signal variance. This added variation can interfere with algorithms/processes that attempt to use ultrasound signal intensity to estimate contrast agent concentration. Additionally, while in any single frame these intermittent/bursty background signals are not significant, when multiple imaging frames are combined to form a composite image, as occurs when measuring tissue perfusion using non-targeted contrast agents, these bursty noise sources can combine to become much more significant artifacts. Note that it is expressly contemplated that the system and method herein can be adapted for use in analyzing and filtering image data in association with the perfusion into tissues of a non-targeted contrast agent. It should be clear that various parameters of the procedure herein can be modified to resolve images containing such agents so as to reduce occlusion and evanescent accumulation of agent between image frames. Hence, while the description references bound or targeted contrast agent by way of operative example, the term should be interpreted to include non-targeted agents where appropriate.

Thus, in one embodiment, a conservative model of background behavior is created from images acquired prior to contrast agent arrival according to the steps below.

First, a composite image is formed by preserving the maximum intensity present in any of the pre-contrast-arrival image frames. In other words, if a pixel is ever brighter than the composite image, the composite image takes on the value of that pixel. This has the effect of preserving any intermittent brightness locations. It also tends to spread any bright spots over space, as the probe and patient tend to be moving during the background image acquisition process, so a single bright spot whose intensity is preserved will have its intensity spread spatially due to the motion.

Then, morphological mathematics are employed to increase the spatial extent of the features present in the background. For example, operations such as dilation, morphological closing, and Gaussian filtering may be performed. In one embodiment, a spatial Gaussian filter with a width of two (2) pixels yields desired results. In this embodiment, the Gaussian filter is applied to the composite image generated by maximum-intensity-projection across the pre-contrast-arrival image frames. Spatial broadening of the background model increases immunity of the subtracted image to probe motion, since background signal will be modeled even if during post-contrast arrival image capture, the probe moves slightly to a different location and/or orientation from that encountered during the pre-contrast frame acquisition process. In other words, the spatial extent of background is intentionally overestimated in this technique.

Optionally, the intensity of weak signals can be selectively increased (local contrast enhancement), thresholded (i.e. anything greater than a small percentage is increased to approximately full brightness), or enhanced via intensity outlier removal techniques such as Matlab's imadjust function, to create an even more conservative estimate. Matlab functions such as imadjust and other Matlab functions referenced herein refer to Matlab version 2017b, and information about these functions can be found in the Matlab 2017b manual.

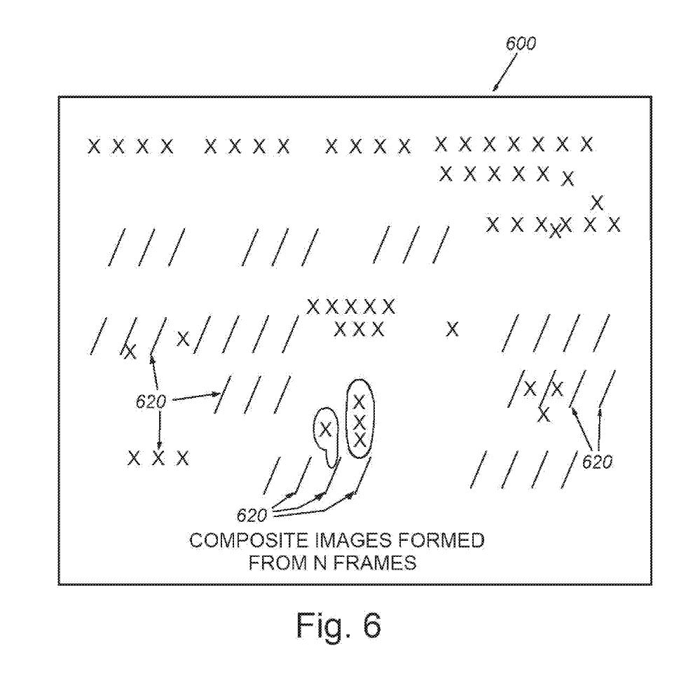

The above-described background modeling technique can be employed in alternate embodiments. For applications, such as early disease detection, in which a false positive is extremely undesirable, utilizing a highly conservative model of background, such as the spatially-enhanced maximum intensity projection described above, is desirable. However, once a tumor location has been ascertained, and the practitioner can then find it desirable to determine its spatial extent, the cost of a false-negative becomes high (as the practitioner wishes to ensure that all of the lesion/tumor has been identified), so he or she may wish to employ a less conservative background model. Such a less conservative approach can include computing the MEAN value rather than the MAX value across a set of pre-contrast arrival image frames, even though this risks some background signal being mistaken for contrast agent signal. Other less conservative options, such as a projection that takes the value beyond which a certain percentage of the frames are brighter, can also be employed. Reference is made to FIG. 6, which shows a schematic representation of a composite image 600 of the tumor-containing region previously represented in in FIGS. 4 and 5. This exemplary image can be formed by data fusion of (e.g.) 16 frames (acquired over the course of several seconds) via Maximum Intensity Projection prior to contrast agent administration. Intermittent background features (e.g. features 620), when combined across multiple frames, can become much more significant at this stage. Hence, the fusion over time in some situations exposes significant spatial structure of the incompletely suppressed B-mode signal.

Background modeling as contemplated herein can employ various hybrid approaches according to alternate embodiments. For example, the intensity of the background signal in the composite image can be used as a prompt for the degree of spatial broadening that may be required. Areas that have a high amount of background signal activity can benefit from additional spatial extent, while areas with a low amount of background intensity, for which the impact of a background estimation error is smaller, can benefit from a lower degree (smaller spatial broadening parameter) of spatial expansion.

(ii) Construction of Background Signal Model Using Images Acquired After Contrast Agent Arrival

To detect background signal, ideally the system and method should include imaging samples acquired prior to the arrival of contrast agent at the site being imaged. The practitioner would ideally maintain the imaging perspective (i.e. not move the patient or the probe) throughout the contrast agent administration process, observing contrast agent bolus arrival and obtaining post-contrast binding images from the same location and imaging perspective. In this manner an example of the background signal intensity present in that region from that perspective is available, and can be used as an example for removal of background signal from the acquired post-contrast images.

However, in many actual clinical examination scenarios, it is not practical to obtain examples of background signal prior to arrival of contrast agent. For example, if the practitioner lacks prior knowledge as to where a lesion/tumor is located, and must scan a significant volume to locate it, then it is not practical to aim the scanner specifically at the tumor location prior to contrast agent injection. The alternative, of acquiring examples of contrast-free images via destruction of microbubbles by a high-energy ultrasound pulse, is in many cases undesirable for use in humans due to concerns about damage to delicate tissues. Thus, employing a technique to differentiate background signal from contrast agent signal, without a contrast-free example, is highly desirable.

A basic technique to differentiate background in the general absence of pre-arrival images is to use the B-mode signal itself as a gating factor. Illustratively, any pixel that exhibits greater than 90% intensity in the B-mode image is more likely to leak through to the contrast-mode image, so could be considered as a likely source of intermittent background. This approach is effective, but is sometimes not sufficient to be of practical use on its own for applications such as screening for diseases, such as cancer.

Alternatively, a technique that is more effective is to image while there still exist freely-flowing microbubbles in the blood stream at the site, but at a lower concentration than were present after the initial bolus arrival. For example, imaging approximately five minutes after contrast agent injection is an effective time point to image for such free-flowing microbubbles. At that time, microbubbles targeting a certain molecule will have effectively bound to their targets, typically in blood vessels, and the flowing microbubbles will be flowing through those same and neighboring blood vessels. This introduces variation into the signal generally. Note that signal due to imaging artifacts and insufficiently suppressed tissue signal are, conversely, not necessarily co-located with blood vessels, and depending on the imaging arrangement, can in fact occlude, or be occluded by, any signal from the circulating microbubbles. In this case, examining statistical properties of the signal intensities can help distinguish between background signal and contrast agent signal.

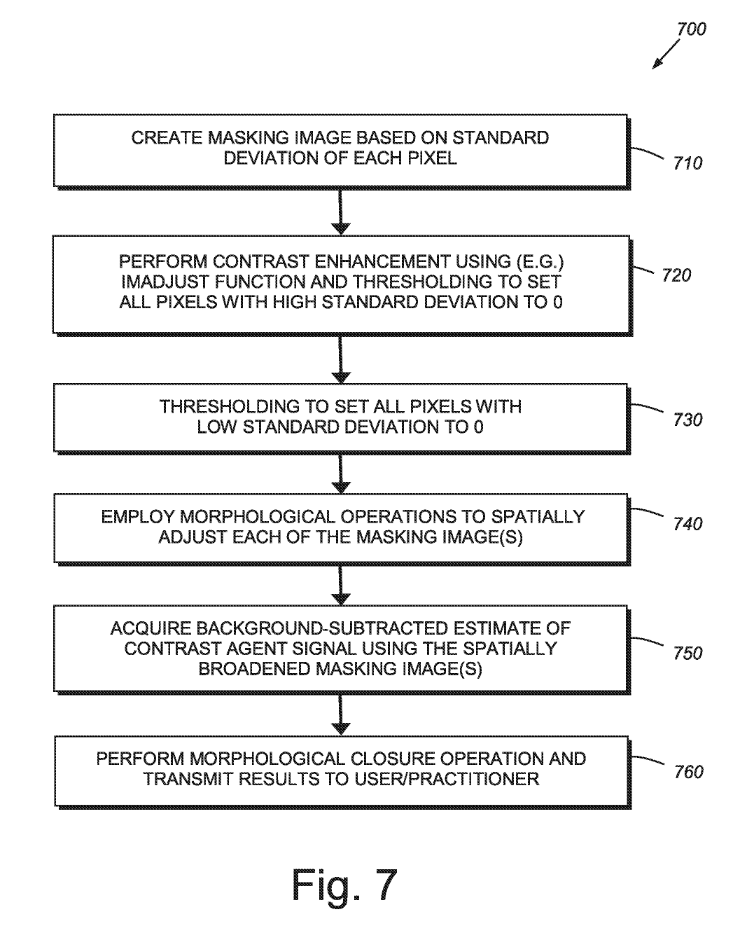

More particularly, it is contemplated that a procedure for background modeling where pre-arrival imaging is absent or insufficient can employ a masking image based on properties such as mean, maximum, minimum, and standard deviation of the intensity at each pixel. Such an approach can effectively generate a useful background model. It can operate in the following manner with reference to the procedure 700 shown in the flow diagram of FIG. 7.

First, in step 710, for each measurement window, the procedure 700 creates a masking image based upon the standard deviation of each pixel in a window. In this embodiment, illustrated using Matlab syntax below, the procedure scales the standard deviation so its minimum value is 0 and its maximum value is 1. Then, in step 720, the procedure 700 performs contrast enhancement of the masking image using the imadjust function, and performs thresholding so as to set to 0 all pixels whose standard deviation is relatively high (e.g. greater than approximately 0.98 in the contrast-adjusted image of standard deviations). This mask eliminates pixels that have extremely high variance, which are likely to come from bursty background and/or from flowing microbubbles that are selectively occluding background as they flow, or from other unknown sources. Notably, it is recognized that accumulated contrast agent exhibits a relatively low variation in comparison to various other sources so this approach leverages this characteristic. The exemplary Matlab syntax is as follows:
High_standard_deviation_elimination_mask=imadjust(imscale(image_of_standard_deviations_within_window))<=0.98);
   where imscale is a function that linearly scales the maximum and minimum intensities of an image into the range of 0 to 1.

Then, in step 730, for each measurement window, the procedure 700 creates another masking image based on the standard deviation of each pixel. In this embodiment, after scaling and contrast enhancement, only pixels whose variation is not amongst the lowest are retained. This thresholding can set to 0 all pixels whose standard deviations are relatively low (e.g. approximately less than 0.05). This eliminates pixels in the image whose intensity arises from sustained background sources, which do not have (are free of) significant variation introduced by the flowing contrast agent. This condition can result when such pixels are not co-located with blood vessels, or can result because of the nature of the imaging artifact (such as resonance) that otherwise generates intensity at that location. The exemplary Matlab syntax for this step is as follows:
Low_standard_deviation_elimination_mask=imadjust(imscale(image_of_standard_deviations_within_window))>0.05;

Next, in step 740, the procedure 700 employs morphological operations, clear to those of skill, to spatially adjust each of the masks. For example, these morphological operations can be implemented with machine vision system recognition and alignment tools, among other software.

Next, in step 750 of the procedure 700, a background-reduced estimate of contrast agent signal is then acquired by utilizing the spatially broadened masks from step 740 above. The procedure step can employ the following, exemplary Matlab syntax in an exemplary implementation:
backgroundCorrectedEstimate=(imscale(imadjust(imscale(window_bound_contrast_estimate) . * . . . max(0,(1-imdilate(1-High_standard_deviation_elimination_mask,strel(‘disk’,1)))) . * . . . (1-imdilate(1-Low_standard_deviation_elimination_mask,strel(‘disk’,7))))));
   Note that for an exemplary screening for pathologies, for example, a cancer screening application, the spatial spreading on the low-standard deviation mask is chosen to be significantly larger than the spatial spreading used on the high standard deviation mask.

It is recognized that, for tumor detection (as opposed to spatial extent evaluation), it is often desirable to perform morphological image enhancement operations, such as closure, prior to presenting results to the user. Thus, in an embodiment, step 760 of the procedure 700 performs morphological closure using (e.g.) a 3-pixel disk structural element this is effective in operation. The exemplary Matlab syntax is as follows:
Final_Bound_Contrast_Result=(imclose(backgroundCorrectedEstimate,strel(‘disk’,3))>0)

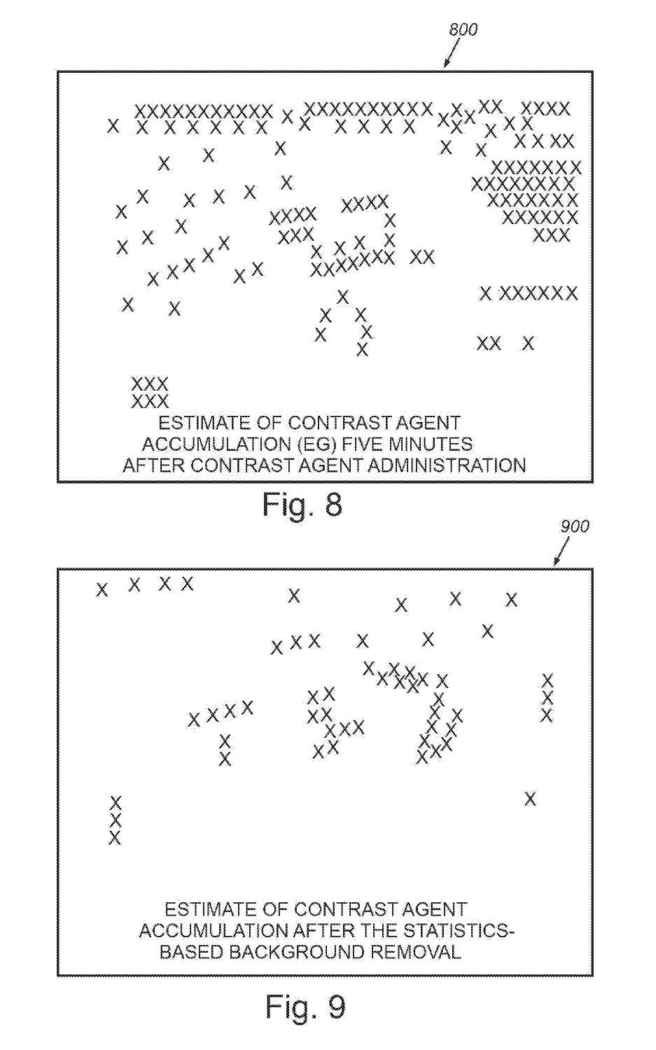

The results of this background removal process, determined in a manner free of any example of a contrast-free background, are shown in the exemplary schematic image representations of FIGS. 8, 9, 10 and 11. In particular, the representation of FIG. 8 depicts estimates of contrast agent accumulation captured via imaging of a certain pathlogy, for example, a tumor, several minutes (for example approximately five (5) minutes) after administration of contrast agent. The contrast agent signals in the representative image 800 are typically confounded by background signals caused by tissue leakage artifacts and other signal sources. Hence the overall image would display a mottled and spotted effect that obscures the delineation of the regions of bound contrast agent associated with lesion/tumor tissue. In FIG. 9, the exemplary representative image 900 depicts estimated contrast agent accumulation after statistics-based background removal (e.g. with no use of examples), and morphological closure. The tissue-leakage, and several artifacts associated with non-molecularly bound contrast agent, would be eliminated by the illustrative techniques, as exhibited by the representation of a somewhat less noisy image 900.

(iii) Additional Processing to Enhance High Confidence Regions

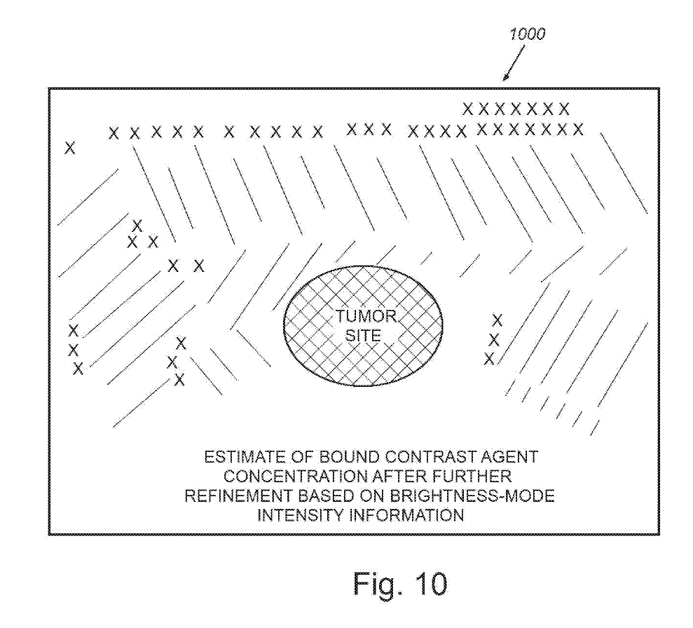

It is recognized that the techniques and procedures described above operate effectively in association with a variety of imaging arrangements. However, they do not remove all background intensity under all conditions. It is contemplated that images of accumulating contrast agent can be additionally enhanced by further adjusting contrast within areas of low B-mode intensity, where background is likely to be lower. This approach effectively de-emphasizes the contrast-mode view of areas of the image that have high B-mode signal, in essence producing an image that highlights areas where accumulation of contrast agent estimates have high confidence, since high background signal in areas of low B-mode signal are less likely to occur. For example, in ultrasound shadow areas, background tends to be very low, so any contrast-mode signal present in those areas is more likely to be valid signal arising from contrast agent rather than background signal. This effect can be exploited by processing the B-mode intensity, optionally combined with a noise reduction operation such as the morphological operation OPEN (opening), to selectively enhance signal within the shadow region (for instance through intensity multiplication), by diminishing signal elsewhere and then rescaling to enhance contrast within the shadow region. FIG. 10 depicts an exemplary representative background-reduced image 1000 of estimated bound contrast agent concentration, that would occur after further refinement based on B-mode intensity.

The following embodiment, with results shown in FIG. 10, can be considered effective for seeing within shadow regions for imaging of pathologies, for example, cancer imaging. The below Matlab code shows the B-mode bound contrast estimate as a dark, shaded region generally within the drawn boundaries, and the background as a shaded region generally outside the boundaries. However, the bound contrast appearance is limited to areas where the morphologically expanded B-mode intensity is less than for example, 40%-45% of its maximum. This results in contrast expansion of the bound concentration estimates within the shadow region, making the bound agent accumulation and hence tumor delineation more visible, as shown in FIG. 10. Using Matlab syntax to describe this embodiment, where the variable B-mode is a single B-mode image frame captured approximately five (5) minutes after contrast arrival, and the variable Final_Bound_Contrast_Result is the background-eliminated result described above. The imthreshold function, as used below, sets to 0 any element of the image that lies outside the range 0.2 to 1.0. The following exemplary syntax can be employed:
Image_of_contrast_accumulation=Final_Bound_Contrast_Result; imadjust(imthreshold(imadjust(imscale(imscale(Image_of_contrast_accumulation) . * imopen(double(imadjust(imscale(bMode))<0.4),strel(‘disk’,7)))), 0.2, 1.0))

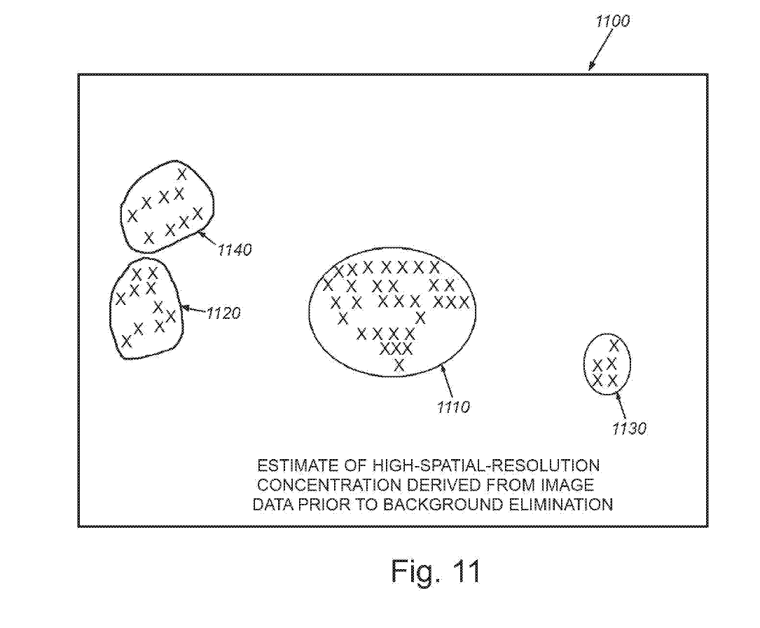

Note that the image representation depicted in FIG. 10 would have limited spatial resolution due to the morphological operations. In one embodiment, rather than displaying this image directly, within areas of low B-mode intensity and hence low suspected background activity, the background-reduced image may be used as a mask to display the higher-resolution concentration estimate image that was present prior to background removal. Again, using the code syntax shown above, but rather than having the Image_of_contrast_accumulation be the result of the background removal process, use the background reduced result being>a threshold (such as 0) as a mask to selectively display the non-background-subtracted result:
Image_of_contrast_accumulation=window_bound_contrast_estimate .* double(Final_Bound_Contrast_Result>threshold);
   In other words, the final image is the estimated bound contrast estimate with background signal included (so that there is not undue loss due to the conservative background model being subtracted), masked to show only places where the background-reduced image had signal greater than a threshold. This masking approach permits the texture/high spatial resolution information that would have been eliminated by background removal and other forms of filtering to remain intact, but only in selected low-background signal locations. This approach is illustrated in FIG. 11, which shows a schematic representation of an image 1100 of the scanned site, in which the regions for which B-mode image has low intensity and the background-subtracted version has intensity greater than a threshold, are used as masks applied to the bound contrast agent accumulation estimates that were derived prior to background elimination. Note that exemplary regions 1110, 1120, 1130 and 1140 of high contrast are shown with boundaries drawn generally around them in this depiction. More particularly, high spatial resolution concentration estimates derived from data prior to background elimination, masked by areas in which the background-reduced signal strength is greater than a threshold (in this case the threshold is set to 0). As an added illustrative filtering effect, the results are drawn here only in regions (e.g. region of tumor site 1110 and other regions 1120, 1130 and 1140) where background is anticipated to be small, due to low intensity of B-mode signal.

In addition to the selective display approach represented in FIGS. 10 and 11, where contrast agent concentration in areas of high B-mode intensity are not shown, it is often desirable to combine via image compositing the higher resolution (non-background subtracted or less filtered) estimates in areas where background signal is likely to be low with the lower spatial resolution estimates that result from filtering in regions of high background signal. The degree of spatial resolution loss can be varied by adjusting the morphology parameters—even to the point of no loss—but at the cost of increased likelihood that background signal will find its way into the resulting images.

B. Occlusion Detection and Compensation

(i) Observation that Accumulating and Flowing Contrast Agent Can Occlude the Tissue Background Signal

It is currently recognized that all existing approaches to the modeling of contrast agent arrival assume that the presence of contrast agent will increase signal intensity. Recognizing that signal intensity may, in fact, decrease in the presence of contrast agent allows for a novel approach to contrast agent analysis by recognizing and exploiting this effect.

Note that additional information relevant to the embodiments herein can be found, by way of useful background information, in Ultrasound Molecular Imaging With BR55 in Patients With Breast and Ovarian Lesions: First-in-Human Results, by Juergen K. Willmann, Lorenzo Bonomo, Antonia Carla Testa, Pierluigi Rinaldi, Guido Rindi, Keerthi S. Valluru, Gianluigi Petrone, Maurizio Martini, Amelie M. Lutz, and Sanjiv S. Gambhir, Journal of Clinical Oncology, Mar. 14, 2017.

Close observation of animal model images and of the published images in FIGS. 1 and 4 of the Willmann et al. reference, reveals previously unrecognized situations in which contrast agent signal replaces (i.e. occludes), rather than adds to, signal from a tissue leakage artifact. This is significant from a practical perspective. The ability to avoid false-positive results, in which background signal is misinterpreted to be contrast agent accumulation, as well as false-negative results, in which contrast agent accumulation is misinterpreted to be background signal, can be enhanced by detecting and accounting for this occlusion effect. Identifying and compensating for the transition between additive and occlusatory behavior of contrast agent is one aspect of the illustrative embodiments herein.

The system and method herein includes processes and techniques, such as over-subtraction detection, to detect and exploit this transition from additive to occlusatory behavior so as to produce improved estimates of contrast agent concentration in tissue. These processes and techniques are applicable to both quantification of molecularly bound contrast agent, as well as to other measurements involving contrast agents, such as measurement of overall blood flow and/or perfusion, which can benefit from accounting for the background occlusion effect to produce more reliable and more accurate results. The methods proposed build on previous work involving detection of occlusion vs. reflection in image backgrounds that were developed for terrestrial imaging applications. By way of further background, reference is made to U.S. Patent Application Publication No. 2017/0352131, published Dec. 7, 2017, and filed as Ser. No. 14/968,762, on Dec. 14, 2015, entitled SPATIO-TEMPORAL DIFFERENTIAL SYNTHESIS OF DETAIL IMAGES FOR HIGH DYNAMIC RANGE IMAGING, by Berlin, et al., which is incorporated herein by reference, and the general teaching of which is incorporated by reference and described further below. In brief summary, this application describes various multi-layer separation techniques to see through translucent objects such as tinted windows, selectively amplifying the fraction of the light at each pixel that was due to the ‘subject’ of the photograph (i.e. a person sitting in a car) without (free of) amplifying light at each pixel associated with optical reflections off of the tinted windows or light associated with the background. In another embodiment, this application describes subtracting the background from an image taken from a video of a subject walking through an environment with a well-lit background. That embodiment solves the problem of subtracting the well-lit background from the image containing the subject resulting in a black hole as the background image is subtracted from the subject. It uses the rate of change associated with motion of the person sitting in the car, or walking through the environment, which differs from the rate of change associated with the reflected objects or background, as well as detection of oversubtraction of occludable background, as prompts to separate out the various sources of light. Applicant has recognized that medical imaging technologies such as contrast-based ultrasound imaging can exhibit occlusatory effects that can be addressed using the principles of this teaching.

A novel contribution of the embodiments herein is the recognition that contrast agent exhibits a mix of occlusion and additive behaviors, depending on the imaging context, and that multi-layer separation methods designed to separate an image subject from both additive and occludable confounding signals can be effectively employed to better expose the portion of the received signals that is due to molecularly bound contrast agent.

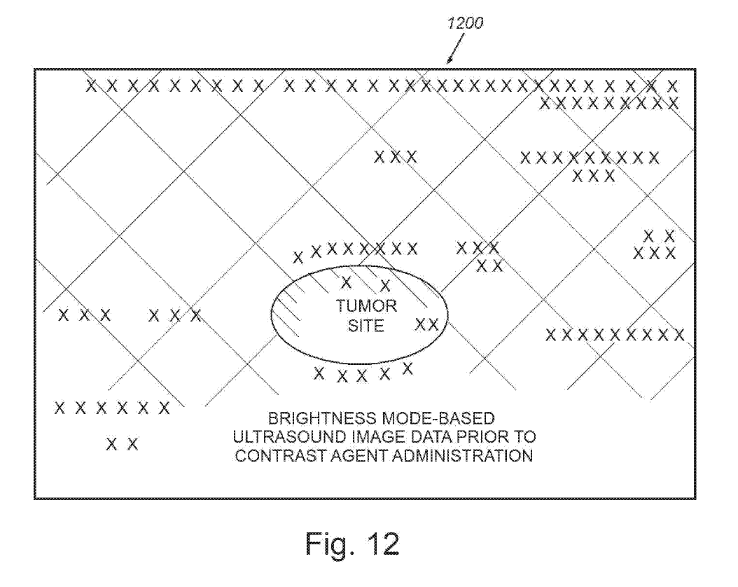

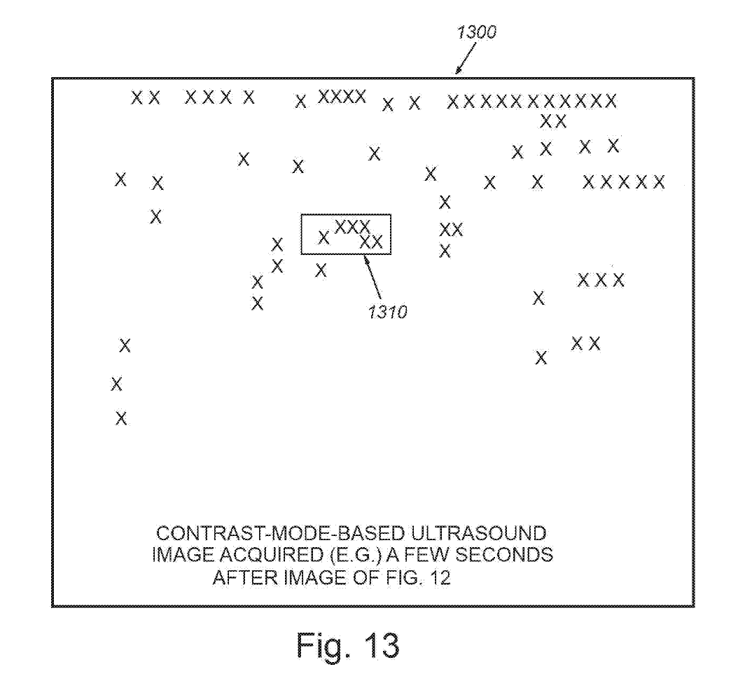

With reference to FIG. 12, a detailed exemplary schematic representation is shown of an image 1200 of tissue acquired using brightness-mode ultrasound imaging administration. All signal present is from tissue and would ideally be suppressed in the contrast-mode view. FIG. 13 is a schematic, representative depiction of a contrast-mode-based ultrasound image 1300 of the same tissue from the same vantage point as that of FIG. 12, acquired a few seconds later, but also prior to contrast agent administration. Note the representation of bright background signals such as tissue leakage signals highlighted (as small cross-hatches) in the rectangle 1310.

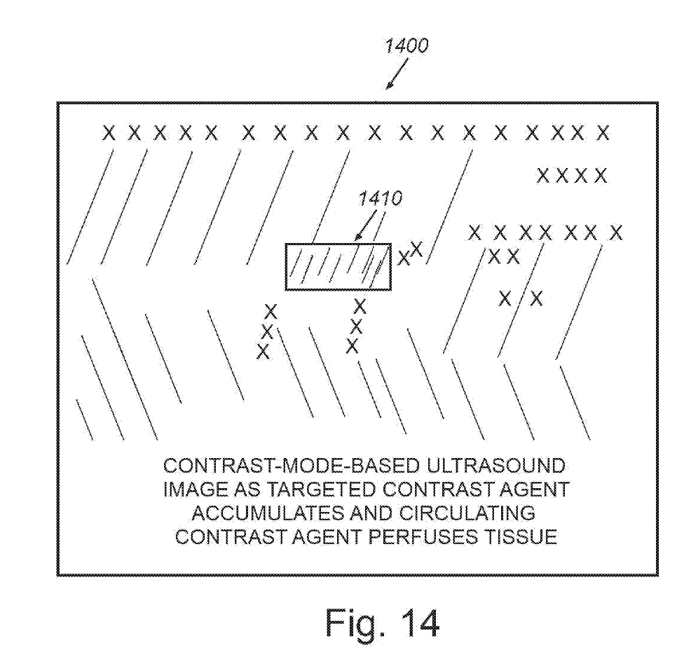

With reference now to FIG. 14, a schematic representation of an image 1400 is shown, based upon contrast-mode ultrasound imaging of the above-described tissue from the same perspective as that in FIGS. 12 and 13 (e.g.) fifty to sixty seconds after arrival of targeted contrast agent. As targeted contrast agent accumulates, and as circulating contrast agent perfuses tissue, in many places the contrast agent increases the intensity of the ultrasound signal. However, the intense tissue background signal that was displayed in FIG. 13 (within the rectangle 1310) is replaced by less-intense contrast agent signal in the same region (rectangle 1410), since in certain locations background signal decreases when contrast agent arrives and occludes the background signal.

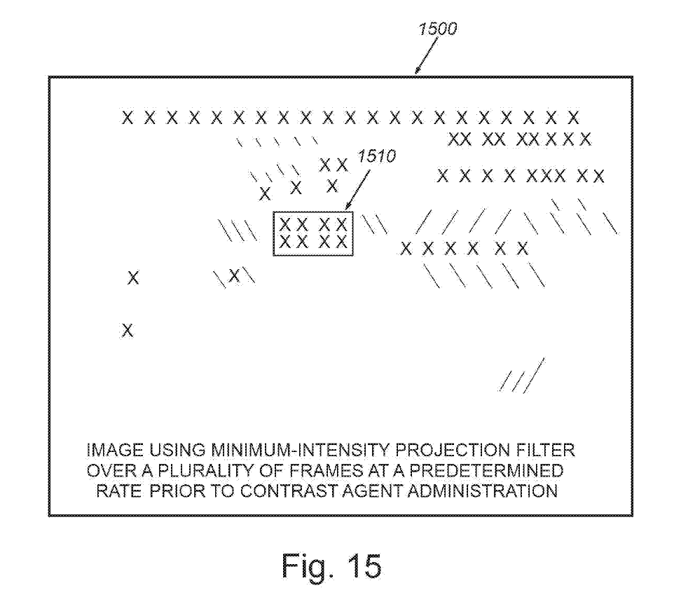

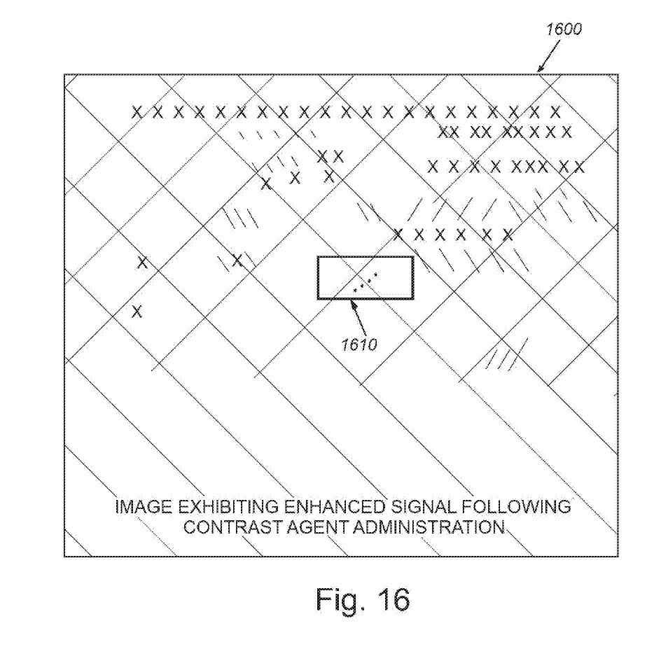

The occlusion effect can be even more pronounced when signal enhancement techniques are employed that combine information from multiple images of the same tissue region. For instance, a minimum-intensity-projection (an enhancement sometimes used to differentiate stationary (bound) contrast agent from flowing contrast agent) over a window of multiple (e.g.) twenty (20) frames yields a pre-contrast agent administration image shown in the schematic image representation 1500 of FIG. 15, and the post-contrast agent administration schematic image 1600 shown in FIG. 16 (with both images would be shown on the same intensity scale of 1-255). In this example, the maximum intensity of the region 1510 would be read as (e.g.) approximately 175 and the maximum intensity of the region 1610 would be read (e.g.) as around/approximately 100. Note that the representation of the post-contrast agent administration/arrival image has less intense signal in the region of the tumor/lesion (within the rectangle 1610) than the pre-contrast agent administration image (within the rectangle 1510). More particularly, in this example, arrival of the contrast agent decreases the enhanced signal intensity in the region (1610) of the tumor by (e.g.) approximately 43%. Thus, as a general effect, the addition of contrast agent actually decreases the multi-frame enhanced contrast-mode signal intensity in the region of the tumor.

(ii) Occlusion Compensated Background Removal

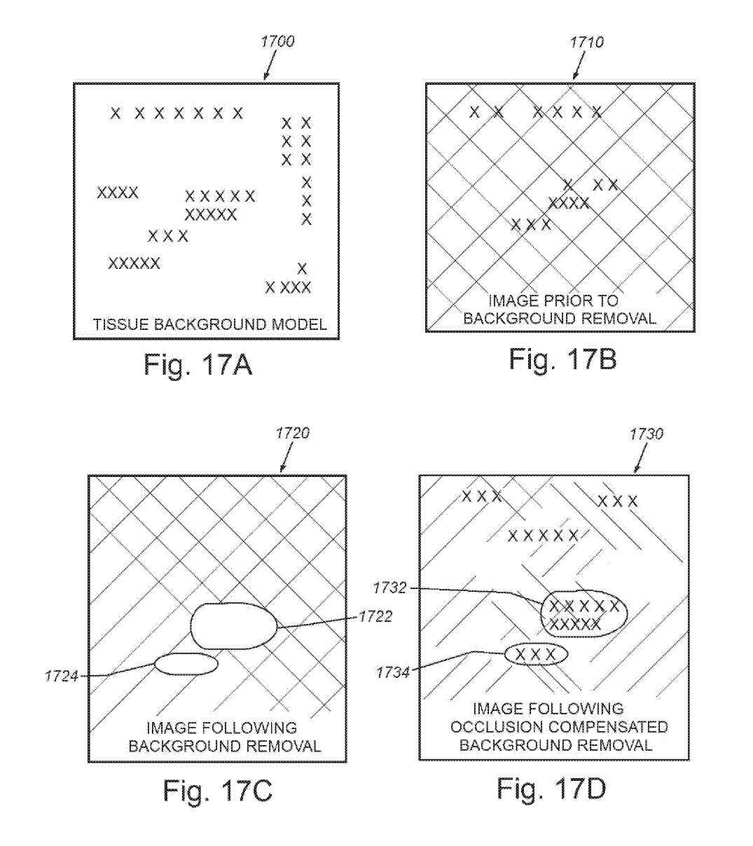

A current technique in the prior art for handling residual tissue signal removal is known as “background removal” or “background subtraction”. In this approach, a frame prior to contrast agent arrival is used to build a model of the background signal, which is then subtracted from later-arriving frames. The basis for this approach is that intensity increase between earlier and later frames is due to the arrival of contrast agent. In the absence of occlusion effects, background removal works effectively. However, in the presence of occlusion effects, background removal leads to unobservable regions, which can be termed, black holes, in which the background signal is larger than the newly acquired signal. In these cases, background removal leads to no signal at all, as shown in the schematic image representation 1720 of FIG. 17C, described further below. In particular FIGS. 17A-17D schematically depict the graphical results of steps in an illustrative process of occlusion-compensated background removal according to an embodiment. FIG. 17A shows a schematic image representation 1700 of the tissue background model, constructed from multiple frames acquired prior to contrast agent arrival. FIG. 17B shows a schematic image representation 1710 of the tissue after contrast agent arrival but prior to background removal, in accordance with the principles herein. FIG. 17C shows a schematic image representation 1720 of the tissue following background removal as described herein—that is, after contrast agent arrival, and after undergoing (e.g.) subtractive background removal. FIG. 17D shows a schematic image representation 1730 of the tissue following occlusion-compensated background removal according to an illustrative embodiment.

Note how the subtractive background removal process (see image 1720 in FIG. 17C) would generate one or more black hole(s) 1722, 1724 in the center of the image due the system/process failing to account for occlusion of the background by the contrast agent. Note also that due to the inversion of the color schemes for schematic illustration purposes, the black holes that would normally appear in a runtime image, are illustrated herein as appear as white holes in FIG. 17C. However, for purposes of the description, the term “black hole” is used to describe this image effect. FIG. 17D shows background removal which accounts for occlusion, and successfully removes the portions of the background model that are acting in an additive manner (thereby simplifying the image) without (free of) removing the portions of the background that have been occluded(which would generate a black hole). In this case, the image being processed is a maximum-intensity-projection across (e.g.) twenty (20) frames, providing an estimate of tissue perfusion. Use of occlusion-compensated background removal (the results of which are shown in the schematic image representation 1730 of FIG. 17D) permits visualization of tissue perfusion even within the region 1732, 1734 that would otherwise have appeared as a black hole.

The above-incorporated Berlin et al. patent application (U.S. Patent Application Publication No. 2017/0352131), describes a multi-layer approach to the separation of multiple sources of signal in an image in a manner that avoids black holes caused by background occlusion. That approach involves dividing an image into the subject layers (representing in that application the object that one desires to observe), the reflection background layers (which refer to non-subject signal that is intermixed with, or added to, the signal associated with the subject), and the true background layers, which are blocked by the presence of the subject. It should be clear that in the 2017/0352131 application to Berlin et al., the true background is occluded by the subject, or person, that the method seeks to image more clearly, while in the present application, the occluding subject can be bound or unbound contrast agent that this method may or may not seek to see more clearly, however, the Berlin application is useful background information for its teachings regarding the subtraction of a background that can be partially occluded.

In accordance with the above-incorporated application, it should be clear to those of skill that such multi-layer separation technique(s) can be adapted and applied to ultrasound imaging. In some imaging situations contrast agent is additive (added to the background signal), akin to reflections on a glass window, and in other situations contrast agent can occlude the background signal, akin to a person moving in front of a background light source. Multilayer separation can distinguish between occlusive and additive signals, so as to avoid subtracting background signal that has already been eliminated from the image by occlusion. In one embodiment, the occluded portion of the background is removed from the background model prior to performing background subtraction, producing an occlusion-compensated background model. This occlusion-compensated background model is then subtracted from the image. Subtracting the occlusion-compensated background model from the image instead of subtracting the full background model from the image can avoid subtracting background that has already been removed by occlusion, avoiding creation of a black hole in the portion of the image where the background had been occluded by contrast agent. Encompassing the multi-layer separation technique(s) makes it practical to overestimate the background tissue signal, on both a spatial and temporal basis, thereby creating black holes. This overestimation helps prevent background signal from being interpreted as accumulated contrast agent, making false-positive results less likely. Relying on the multi-layer separation technique to detect and correct for this overestimation, overcoming the black holes, permits the overestimation technique to be used effectively without unduly suppressing the contrast-agent signal.

With further reference to the above-identified U.S. Patent Application Publication No. 2017/0352131 to Berlin, medical imaging technologies, such as those derived from ultrasound and other forms of radiation-based imaging, can exhibit effects similar to the optical effects described in the application. That is, reflected energy from overlying masses such as contrast-agent laden blood vessels partially, but not completely, obscures objects/features of interest, such as tumors. Underlying objects can also provide a signal that is partially obscured by accumulation of contrast agent in a feature of interest, such as a tumor. The resulting images are thereby a combination of the features of interest and other features that are undesired and confuse the overall view of the diagnostic region.

In an embodiment, the Published Berlin Application's described method for separating reflective background, a subject, and a true background that is partially occluded by a subject can be applied to optimize a contrast enhanced image of a tumor or other structure of interest within the body, in the presence of partial occlusion of the background by flowing or stationary contrast agent (and/or other obscuration). In an embodiment, a video of a person walking through an environment having an occludable background and reflective background can be separated so that the image of the moving person can be isolated. Isolating the image of the person can be done by subtracting the reflective background and the background from the image. However, when the background includes well-lit features, subtracting the entire background from the image containing the person can result in the creation of a black hole in the background-subtracted image. When the person is moving in front of a well-lit portion of the background, subtracting the light intensity of the background from the image containing the person can result in pixel intensities of less than zero in locations where the presence of the person blocks the background light, since one is subtracting light that is no longer present in the image. That application describes separating the reflective background, the moving subject, and the true background by applying statistical analysis to the pixels in a measurement window of frames. The algorithm developed for the subject isolation, described in U.S. Patent Application Publication No. 2017/0352131, may be applied to imaging of signal arising from a tumor mixed with signal arising from a surrounding flowing contrast agent, with the accumulating and in some cases the flowing contrast agent acting to block, or occlude, the background. When applied to ultrasound, the HDR image composition module may incorporate images captured using alternative imaging modalities, such as B-mode rather than contrast-mode ultrasound, to place the contrast-mode tumor image in the context of the B-mode organ structure.

Before contrast agent is introduced, all pixel intensity is due to background. After contrast agent arrival, any decrease in pixel intensity is due to contrast agent in front of, or occluding, the background. If a decrease in pixel intensity lasts longer than approximately a few seconds, the long decrease in pixel intensity can indicate that the background has been occluded by bound contrast agent. A slow growth in pixel intensity at that location can indicate that bound contrast agent is accumulating at that location, and may indicate the presence of a tumor. Pixel intensity at this location can be removed from the background, so that subtraction of the background will not decrease this pixel intensity that is due to bound contrast agent. On the other hand, if a decrease in pixel intensity lasts only momentarily, and pixel intensity quickly increases back towards background intensity levels, this can indicate that the background was briefly occluded by flowing contrast agent, that the occlusion has passed, and that the pixel intensity has returned to background levels and should be removed. Similar to the Berlin application, the method can use the duration of background occlusion as a prompt to promote light from the reflective (additive) background layer to the occludable background layer. Further, the method can maintain different occludable background layer models for each type of occlusion. Background occluded by the flowing contrast agent is occluded on a momentary time scale, and background occluded by bound/accumulating contrast agent can operate on a different time scale. Areas of the background deemed to be occluded by bound contrast agent can be semi-permanently removed from the background model, while areas of the background that are occluded briefly by flowing contrast agent can be removed only momentarily from the background model while they are occluded. This brief removal of background occluded by flowing contrast agent can make the blinking flow of flowing contrast agent become visible. The method can also have intensity thresholds, for example, any pixel with an intensity that is within approximately 5% of the initial pre-contrast pixel intensity can be considered background, since accumulating contrast agent may not be as bright as the background. In this way, the ultrasound images can be separated into areas of bound contrast agent that are areas of interest, areas of temporary occlusion, and true background, so that the background can be subtracted without reducing the pixel intensity of the areas of interest. In various embodiments, the method can color code the areas where background removal was modified, to show estimates of where bound contrast agent is suspected and estimates of where flowing contrast agent is suspected. This can permit the user to review these areas carefully to judge whether the interpretation is correct.

Many techniques for performing HDR image fusion and tonal mapping continuity are known to those of skill in the art of machine vision and associated literature. Many are based on optimizing the contrast of the image, combining images taken under different exposure conditions. Some recent work has focused upon HDR in video images, primarily from the perspective of ensuring continuity of the HDR merging process across multiple frames, so as to avoid blinking or flashes introduced by changes in HDR mapping decisions from frame to frame. However, the above procedure also advantageously teaches optimization of the HDR image tonal-mapping process based on motion of a subject relative to its background image.

More particularly, the illustrative embodiment can utilize the above techniques to separate out various different anatomical components that have been mixed together/confounded in the ultrasound image sequences. In particular, it is thereby possible to disambiguate flowing contrast agent from the tumor and from other objects, based in part on the way that these flowing contrast agent features change/move, in image sequences acquired during contrast-based ultrasound (or similar types) of imaging. Since the image portions due to the flowing contrast agent can change at a different rate from those due to the tumor, it is possible to use the differential in motion rates and/or differential in the rate of change of each pixel's intensities, to estimate how much of the energy captured at each pixel is due to the flowing contrast agent, and how much is due to non-flowing, accumulated stationary contrast agent that can indicate a tumor. The signal associated with an object of interest, such as a tumor, can then be selectively amplified or isolated. Other prompts, such as brightness and texture, can be utilized as well to further disambiguate the various anatomical structures that contribute to the confounded image. Finally, techniques such as amplification and contrast stretching may be employed prior to motion analysis, to make the motion more visible, and also following motion analysis to selectively enhance the portion of the signal that is associated with the object of interest.

(iii) Compensating for Occlusion

It is desirable to detect the transition from additive to occlusion behavior of contrast agent relative to background to appropriately process images in accordance with the illustrative embodiments herein. For example, if the background is initially very intense, then the acquired images should all be very intense, since they incorporate the background. If the acquired images become significantly less intense, then that is an indication that the behavior of the background has changed. This can be detected by monitoring the occurrence of over-subtraction, i.e. situations where the intensity of the acquired image transitions from being at least as bright as the background signal, to being significantly lower intensity than the intensity of the expected background signal. In an embodiment, a thresholding mechanism can be used to determine the amount of over-subtraction, beyond which occlusion of the background is deemed to have occurred, i.e. if over-subtraction_amount>over-subtraction_threshold, then occlusion is present.

Additionally, the time behavior of the intensity can also be considered. For example, the system can require that the intensity fall below a threshold, and remain there for a minimum period of time, in order to be interpreted as the start of an occlusion mode of operation, i.e. if intensity<time_threshold for time T, then occlusion is present. In addition to absolute metrics such as over-subtraction threshold and time thresholds, one can employ statistical measures across multiple time windows, such as a change in the mean of the signal, and/or a change in the standard deviation or the coefficient of variation of the signal over a time interval of interest.

Also, spatial relationships can be employed to disambiguate between local measurement noise-induced reduction in intensity versus an occlusion-induced change. For example, requiring that all pixels within a given radius experience and maintain a reduction in mean intensity within (e.g.) five (5) seconds of one another would provide a potential technique to exploit spatial correlation.

Once occlusion is detected, a compensatory response can be utilized to account for the detected occlusion. One approach is to maintain a three-layer model, consisting of the intensity arising from contrast agent (the subject layer), intensity due to occludable background (which vanishes as contrast agent arrives), and intensity due to the additive background.

For a pixel (or more generally a voxel) at location (x,y) having intensity I; when over-subtraction by an amount alpha (α) is detected at location (x,y), the amount a is promoted from the additive layer to the occludable background layer, and when background removal is performed, a three-layer computation is then executed as follows, where ‘Overall Background’ refers to the combination of occludable and additive background:

Result Image=(Raw Image−Overall Background)+Occludable Background

Incrementing the occludable background model at the location (x,y) by an amount α removes the black hole effect, achieving 0 intensity at the current moment in time. This makes future growth of intensity at location (x,y) become visible without (free of) being masked by background removal.

In some circumstances, additional correction beyond the amount a is desirable. For example, in a circumstance, such as depicted in FIG. 17C, in which full occlusion of the background is occurring, promoting the amount of the background model to the occludable background layer model will cause the full intensity I to be preserved in the result image, i.e. background subtraction effectively does not occur at location (x,y), as it is fully compensated for by addition of the occludable background.

Many variants of this technique are possible, such as maintaining a binary map of pixel locations that have been occluded, and hence should undergo background removal. Alternatively, dynamic filtering models, such as the exponential decay filters described in the above-incorporated Published Patent Application, can be utilized to continuously update the background model.

In circumstances that implicate multiple time points and/or spatial points, image and video quality parameters (such as intensity histograms) can be employed that model the anticipated behavior of the background (for example, as obtained by statistical measurement prior to contrast agent arrival) and compare it to the actual behavior (for example during contrast agent arrival). Disappearance of the background intensity histogram features from the acquired images is used as an indication of a transition from additive to occludable background behavior in an embodiment. Other metrics useful for this purpose include texture information represented in wavelet representations such as the DB4 wavelet, and texture information available from spatial frequency information as is available from (e.g.) Fourier analysis.

(iv) Sources of Occludable Behavior Patterns

The term “occlusion” as used herein refers to (partial or full) replacement of the background signal intensity at a location (x,y) with a signal intensity that corresponds to the presence of contrast agent at location (x,y), i.e. the confounding signal is fully (or partially) removed. There are several possible scenarios that can cause this transition, some of which are associated with local events (such as arrival of contrast agent at the location of interest). However, the transition from additive background to occludable background at location (x,y) may occur due to events that occur elsewhere. For instance, arrival and/or accumulation of contrast agent at a distant location can alter acoustic impedance in a way that introduces or removes a large-scale imaging artifact (such as mirroring or reverberation) that in turn impacts the visibility of contrast agent and/or background signal at location (x,y).

Other physical changes, such as patient motion, ultrasound probe motion, and accumulation of contrast agent (which can scatter acoustic signals) in the volume of tissue that lies between the acoustic probe/transducer and the location (x,y) can also cause transition from additive to effective disappearance/occlusion of background signal. Out-of-plane changes (changes to z) of the imaging slice can also cause background elements to enter and exit the field of view, having an occlusion-like effect in which a piece of background signal appears and/or disappears.

In an embodiment, it is contemplated that the decision of whether background has been occluded can be made on a frame by frame basis, varying at each time sample, or on a time window—by-time window basis. This is appropriate when compensating for background occlusion induced by patient motion—for example, where an object of high background intensity is moving in and out of the pixel/voxel/region of interest. In other situations, such as monitoring the accumulation of contrast agent over time, it is desirable to have the occlusion determination remain in place over time, i.e. once a pixel/voxel has been identified and/or designated by the system as occludable, it can be advantageously treated as occludable, even if the intensity increases back to its original level. This permits monitoring of additional contrast agent accumulation without interference from background removal, once the threshold where occlusion of the background begins has been reached. In contrast agent-accumulation imaging situations, background artifacts infrequently (if ever) reappear once the transition from additive to occludable background has occurred.

Finally, while the description of background occlusion detection and compensation presented above is tailored to arrival of contrast agent, it is notable that these techniques can be equally well applied to monitoring of destruction of contrast agent. For example, a high-energy ultrasound pulse can be used to pop the microbubble contrast agent particles, exposing the background signal. Existing techniques look for image pixels that become darker when the microbubbles are popped, and use the change to estimate what the concentration of bubbles must have existed prior to their destruction. However, noting the presence of background signal, either through pre-contrast arrival monitoring thereof, or through computationally detecting that the signal grew in strength when the bubble was destroyed, can permit more accurate estimates of pre-destruction bubble concentration. Specifically, areas that would otherwise have been ignored as not having a sufficient intensity decrease when bubbles are destroyed, can be evaluated as having bubble presence at a concentration corresponding to the full intensity prior to bubble destruction (in the event that occlusion is encountered) or could be explicitly tracked as ‘uncertain’ rather than treated as ‘positive’ or ‘negative’ results.

While the systems and methods herein are described in terms of pixels or voxels, in alternate embodiments, multi-scale image representations can be employed. For example, pixels can be grouped into clusters to form a varying-resolution hierarchy of images. The above-described techniques can be applied to all levels of the hierarchy, or only to selected levels. For instance, this can focus on local or regional effects while ignoring global effects. The hierarchical representations can include phase representations, hierarchical clustering, pyramid-based representations, triangulation-based representations, and/or other multi-scale techniques known to those of skill and described in computer vision literature.

C. Measurement Window Image Data Fusion and Multi-Window Refinement

The procedure 200 receives a sequence of background-removed windows 244 from step 240 above, and now performs step 250. The step includes the following processes:

(i) Detecting Accumulation of Bound Contrast Agent During High Flow of Unbound Contrast Agent

The illustrative embodiments herein provide techniques that enable monitoring of accumulation of molecularly bound (stationary) contrast agent even in the presence of a substantial concentration of flowing contrast agent, as occurs during and shortly after arrival of a contrast agent bolus at the imaging site. Most existing approaches to non-destructive monitoring of ultrasound microbubble contrast agent accumulation operate based on data acquired several minutes after contrast agent introduction, when the concentration of the flowing contrast agent in the bloodstream has largely subsided. This enhances the ability to measure stationary contrast agent (which is a relatively constant signal) without undue interference from the signal associated with flowing contrast agent. However, the signal from the bound contrast can deteriorate significantly over the course of the waiting period, as bound contrast agent ‘unbinds’ and is released into circulation, and as bubbles self-destruct. Imaging early is also advantageous because the pre-contrast background model will be more recently acquired, and hence, more accurate. Thus, there is substantial advantage to imaging early, while the concentration of bound contrast agent is still high.

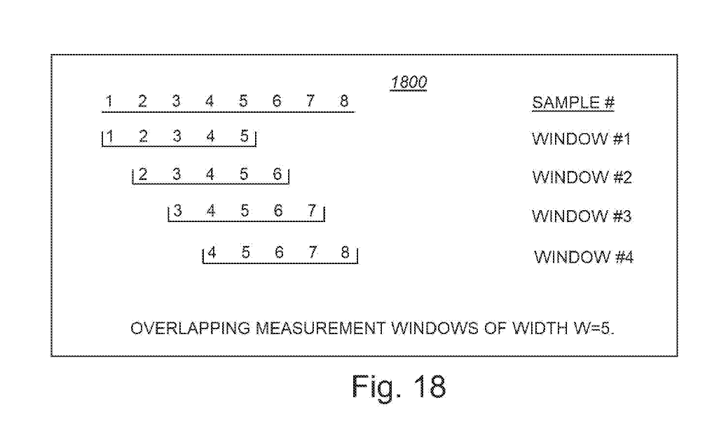

Thus, illustrative embodiments herein can operate to group adjacent data samples into a sequence of overlapping time-based windows, performing statistical analysis of the samples within each window, and then performing cross-window optimization and refinement. FIG. 18 shows a table 1800 of an exemplary measurement window structure, similar to that described above with reference to FIG. 3. For example, window #1 contains samples 1, 2, 3, 4 and 5, while Window #3 includes samples 3, 4, 5, 6, and 7, etc. Grouping samples into windows is advantageous because it permits analysis of samples over relatively smaller time scales during which key parameters such as mean signal intensity are more uniform than is the case over larger time periods. In an embodiment for monitoring bound contrast agent accumulation during bolus arrival, window size can be set to, for example, W=20 at a sample rate of 1 sample per second, or W=80 at 4 samples per second.

In the absence of measurement error or noise, for low concentrations of non-stationary contrast agent, the MINIMUM intensity projection across the samples in a measurement window reflects the portion of the signal that is due to stationary contrast agent particles. This is because in a situation where the concentration of flowing contrast agent is low, it is likely that the measurement window will include a sample for which no flowing contrast agent particles are present at a particular pixel/region, i.e. a moment at which only signal intensity due to stationary contrast agent signal is acquired. In fact, at a sufficient long point in time after contrast agent injection prior to measuring, flowing contrast agent concentration has decreased sufficiently that there will likely be several such samples within a measurement window. The availability of multiple valid samples without flow permits the use of alternative projections that are more resilient to measurement noise, such as the 20% projection suggested in the IEEE 2015 publication entitled Quantification of Bound Microbubbles in Ultrasound Molecular Imaging, as referenced above, which takes the intensities of the weakest 20% of samples to be reflective of the concentration of bound contrast agent.

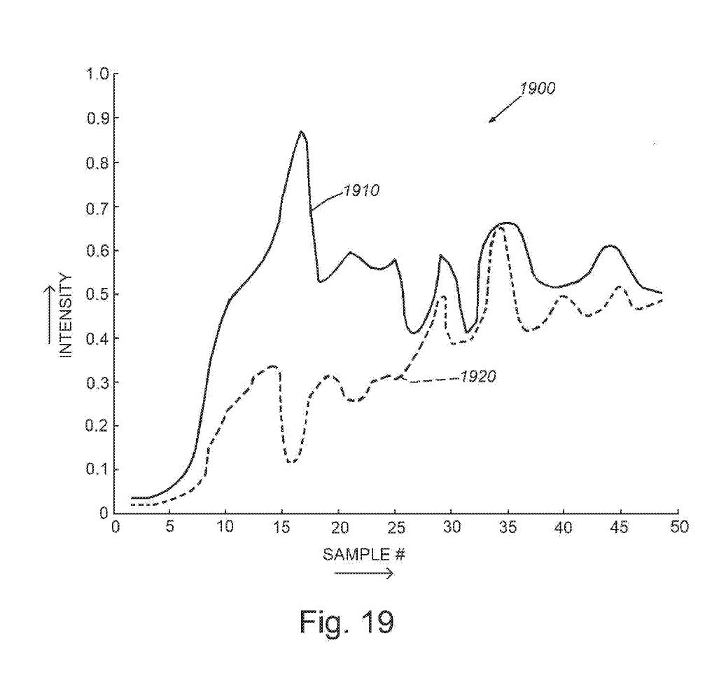

While the above-described approach can effectively handle low concentrations of flowing contrast agent, in high concentration environments, it is not practical since there is likely to be flowing contrast agent present in many, in some cases all, of the acquired samples within each region. Thus, the MINIMUM intensity projection no longer reliably reflects only the stationary contrast agent signal, but also includes some of the flowing contrast agent signal. Using the 20% intensity projection in such circumstances is unreliable, since if finding even one sample with no flowing contrast agent is difficult, then finding 20% of the samples without flowing contrast agent is highly challenging or impractical. Hence, there is a long felt need to develop methods that can estimate stationary contrast agent concentration even when it cannot be measured directly. As illustrated in the representative graph 1900 of FIG. 19, in the presence of a significant time-varying flow of contrast agent particles, it may not be apparent from examination of the raw data what portion of the intensity is due to stationary particles. In the graph 1900 the expected response curve from raw ultrasound data is represented, showing contrast agent signal in an exemplary pathology, for example, a cancer lesion vs. normal tissue. The curve 1910 of the data reflects an exemplary normal region that has high blood flow (and hence high flowing contrast agent intensity) but low accumulation of bound contrast agent. The curve 1920 shows a tumor region that also has high blood flow and that does undergo accumulation of contrast agent due to molecular binding. It should be clear to the reader that it is very challenging to distinguish between these curves by looking at a raw data representation.

(ii) Estimating Stationary (Bound) Contrast Signal Intensity Within Each Measurement Window

It is contemplated that use of statistical models of the flow permits the concentration of bound contrast agent to be estimated even without direct observation of the minimum value. A model-based approach according to an illustrative embodiment relies on statistical properties across the entire collection of samples within the measurement window, rather than relying on any individual sample's value. In an illustrative embodiment, the stationary contrast agent intensities at each location s are modelled in terms of standard deviations below the mean at that location:

s=max(0, u−σ) (Equation 1: mean adjustment approach),

where σ is the standard deviation of the intensity of the samples within the window and u is the mean intensity within the window.

This mean adjustment approach is advantageous because mean and standard deviation are properties computed from consideration of all samples within the measurement window. This reduces sensitivity to a single noisy reading (which can produce a false minimum value). More significantly, this mean adjustment approach does not require availability of any individual sample in which no flowing contrast agent is present, so is suitable for situations involving high concentrations of flowing contrast agent.

Use of the max operator in Equation 1 above effectively eliminates (i.e. sets to 0-intensity) regions whose mean intensity is not at least alpha standard deviations above zero. For example, this operator eliminates pixels that have occasional strong peak signal (and hence high standard deviation), but have very low mean. In some implementations, depending on the nature of the noise in the system, it may be desirable to reference the max operator to a value other than 0. This can be done by subtracting a threshold value τ from u and then (optionally) restoring τ units of intensity to s:

s=τ+max(0, (u−τ)−ασ) (Equation 2)

It should be clear to those skilled in the art that alternatives to the subtraction approach described above can be employed, such as a ratio-based approach that involves scaling of the mean intensity by the standard deviation:

s=τ+max(0, (u−τ)/σ) (Equation 3)

It is contemplated that for imaging of pathologies, for example, a cancer imaging using molecularly-targeted contrast agent particles, during periods of high flow, using logarithmically-adjusted ultrasound data as the input for analysis, the mean adjustment subtraction approach (Equations 1 and 2) will be highly indicative of cancer presence, far more so than is the ratio-based approach (Equation 3), due in part to the aggregation associated with measuring multiple particles simultaneously, as predicted by the central limit theorem.

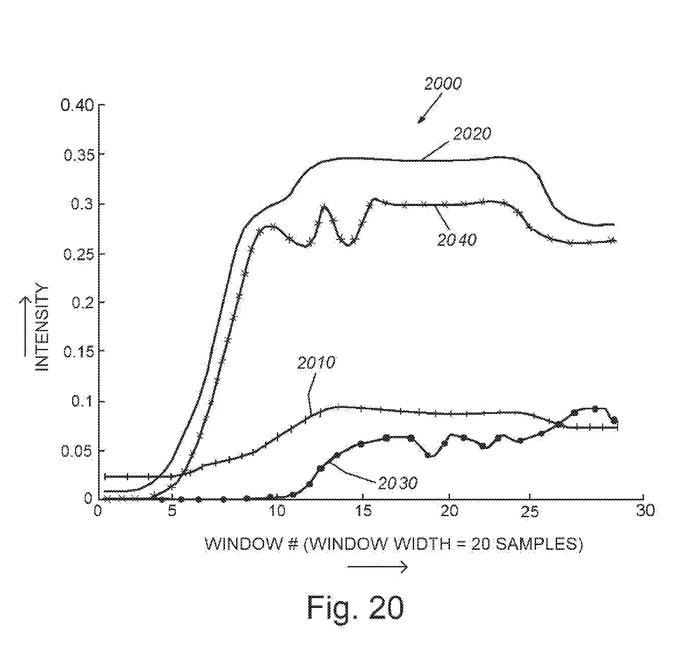

FIG. 20 is a representative graph 2000 (described further below) showing exemplary estimates of the intensity due to bound contrast agent for each window, for both a pathology, for example, a cancer tumor area and, also by way of example, a normal tissue area, generated using the exemplary raw data from the graph 1900 of FIG. 19. In this case, there is a period of very high flow (FIG. 19 samples 20-30, FIG. 20 windows 1-10) as the bolus of contrast agent first arrives, followed by a decrease in concentration of flowing bubbles to a more moderate level. Results are shown for both the MINIMUM intensity projection methods, as well as for the mean adjustment subtraction approach (Equation 1). Note that the MINIMUM intensity projection does not provide fine time resolution on intensity changes, since in a constant or decreasing flow environment, once a minimum value is achieved, it remains the minimum value for several consecutive windows, typically as many windows as a window is wide (i.e. window width W). So even if further binding is occurring, it is typically not visible in the MINIMUM intensity projection measurement until W samples after the previous minimum was encountered. Conversely, the mean adjustment approach estimates the concentration based on all samples within a window, so it is able to change dynamically, providing greater time-resolution on the visualization of binding dynamics. This is quite significant in arrangements where a model of binding dynamics or its parameters is to be fit to the data in a later analysis stage.

(iii) Selection of the Parameter Alpha (α)

The alpha (α) for each portion of an image (such as a pixel or group of pixels) can be customized, or a single value of alpha can be employed across the entire image. It is contemplated that it is practical and effective to use a single value for alpha across the image. This value can be derived in several ways, depending on the embodiment and needs of the application. For example, in applications that are constrained to make conservative estimates to avoid false positive results, such as for early detection of disease, for example, of cancer, the value for alpha can be chosen somewhat larger than in cases where the goal is to prioritize avoidance of false negatives, as when estimating the spatial extent of a tumor for potential surgical removal or treatment. Some of the options for selecting alpha include:

(a) a model, such as the binomial distribution or Poisson distribution, of the flowing contrast agent can be employed, with alpha chosen so as to best fit the model.

(b) Alpha can be chosen to best match the MINIMUM intensity projection at each pixel at a time of modest flow, when the MINIMUM is more likely to reflect the concentration of bound contrast agent than is the case during periods of high flow. For example, alpha (α) can be determined using a time window that ranges from 30-seconds to 50-seconds following arrival of the bolus of contrast agent, after the initial burst of intensity from bolus arrival has subsided and the mean intensity has begun to decrease.

(c) Alpha can be intentionally slightly overestimated to reduce the chances of a false positive result. For example, it is contemplated that selecting α=2.5 will result in a signal that is asserted only where substantial contrast agent has accumulated, while it is expected that selecting α=2.0 will generate results that are comparable to taking the MINIMUM intensity projection for modest-size windows (e.g. W=20 frames).

(d) Alpha can be selected on a per-pixel basis, for example by using a reference window to match the MINIMUM intensity to the mean-adjusted intensity via the mathematical relationship,

Alpha=(pixel_mean−pixel_min)/(pixel_standard_deviation)

within the reference window.

(e) Rather than selecting alpha on a per-pixel basis, alpha can be selected based upon the overall image properties of all pixels that have substantial intensity, which includes both areas of accumulation and areas of non-accumulation. For example, alpha=mean_value_across_all_pixels of the per pixel computation:

Alpha=(overall_mean−overall_min)/overall_standard_deviation.

Use of the statistical approach can provide finer granularity of data with respect to contrast agent binding, with updates occurring continuously rather than a single MINIMUM value persisting over the course of W measurement frames. This continuous updating exposes more of the contrast agent binding dynamics, making it more effective to apply analytical models of contrast agent binding dynamics across window boundaries, computing key indicator variables such as maximum slope, time of arrival, etc.

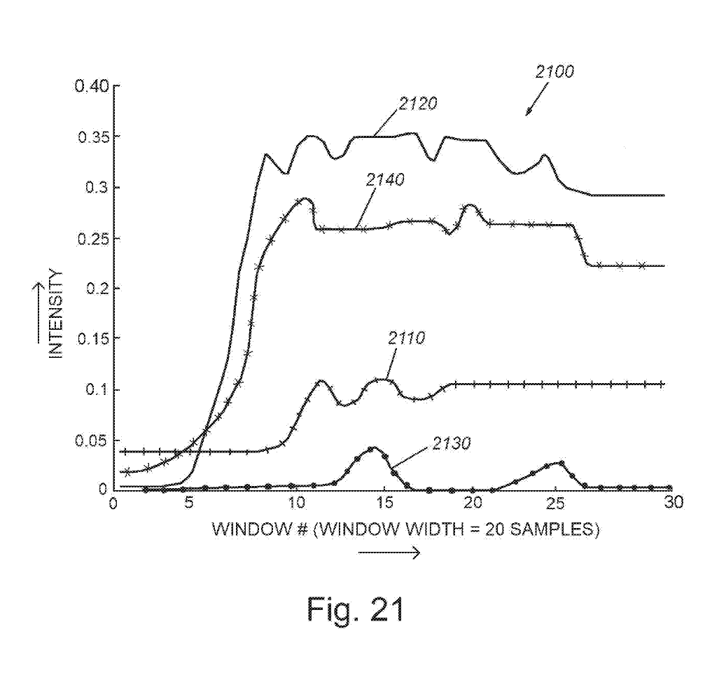

Reference is made again to the graph 2000 of FIG. 20, which illustrates estimates of intensity in each measurement window that is due to bound (stationary) contrast agent, using alpha=2.0. This graph employs the same raw data that is depicted in FIG. 19. Results are shown for both the MINIMUM intensity projection approach (curve 2010 is for normal tissue and curve 2020 is for tumor tissue), and for the mean adjustment statistical approach shown in Equation 1, for alpha=2.0 (curve 2030 is normal tissue and curve 2040 is tumor tissue). Note how during the period of high flow between window 10 and window 20, the MINIMUM intensity is inflated by the presence of intensity associated with flowing (rather than bound) contrast agent in the acquired samples, and then decreases as the flow decreases (i.e. a clear indication that a portion of the MINIMUM measurement was due to flowing contrast agent). For alpha=2, the statistical approach also has somewhat of a dependence on intensity associated with flowing contrast agent, illustrating the desirability of choosing a larger value of alpha that will more fully remove the effect of flowing contrast agent, as shown in the graph 2100 of FIG. 21, described below. This graph 2100 also depicts curves of bound concentration estimates using the MINIMUM intensity projection approach (curve 2110 for normal tissue and curve 2120 for tumor tissue) versus estimates using the statistical approach (curve 2130 for normal tissue and curve 2140 for tumor tissue). Briefly, in comparing the examples in graphs 2000 (FIG. 20) and 2100 (FIG. 21), the intensity contrast ratio between signal in the tumor region and signal in the non-tumor region increases substantially from 3.0× to 10× as alpha increases from 2.0 to 2.5.

An advantage to the statistical approach is that its sensitivity can be adjusted to be more conservative in the reporting of bound contrast agent presence. For example, increasing alpha to 2.5 yields the results shown in the graph 2100 of FIG. 21, in which the final estimate of accumulation in the tumor area (approximate intensity of 0.23 for window #30 in curve 2140 of FIG. 21) is significantly distinguished from the estimate of accumulation in the non-tumor area (approximate intensity 0.02 for window #30 in curve 2130 of FIG. 21), a factor of approximately ten-times (10×) contrast ratio between tumor and non-tumor. In comparison, the MINIMUM approach yields a significantly smaller contrast ratio of 0.27 (tumor curve 2120) versus 0.08 (non-tumor curve 2110), a factor of approximately three-times (3×) contrast ratio.

Notably, the value of model parameters such as alpha (i.e. alpha=2.0 vs. alpha=2.5), supplied herein are illustrative. In general, selection of specific values for these parameters will depend on the dynamic range of the ultrasound system, the image acquisition parameters (such as power levels, etc.), on data pre-processing methods, and on properties of the ultrasound probes utilized in acquiring the measurements. Referencing the selection of alpha to acquired properties such as window MEAN and window MIN, both within a pixel and across an image, as described above, can be used to calibrate, or dynamically adjust, these parameters.

Images generated in accordance with the minimum intensity and statistical mean-adjustment techniques described above (for example, window #30 after subtraction of background signal) using alpha=2 (a moderate estimate) would have fairly comparable appearances. However, even at the moderate flow rates of window #30, the minimum intensity projection would display more low-level noise artifacts in the non-tumor areas due to its sensitivity to measurement error in the single data point that is the minimum. This is the case for images that have not yet had morphological filtering or cross-window optimization performed (described below). Where a conservative estimate (alpha=2.5) is employed, the minimum intensity image will show significantly more intensity regions than the statistical approach image—again, before morphological filtering or cross-window optimization are performed—thereby creating more opportunities for potential false positive results.

(iv) Alternatives to the Mean-Subtraction Model

For imaging of pathologies, for example, cancer imaging, it is contemplated that the mean-subtraction model operating on a window-by-window basis with a common value for alpha across the windows can be employed effectively. Depending on the characteristics of the tissue in the region being imaged, a variety of alternative models can be utilized. For example, a simple model based on the binomial distribution can be used, in which the cross-sectional flow through a vessel may be modeled as consisting of N compartments, each of which may be permanently occupied by stationary contrast agent particles, or which may be left ‘unbound’ to act as a host to flowing contrast agent particles passing through that cross section. Specifically:

U=the # of unbound compartments;

B=the # of bound (permanently occupied) compartments;

N=total # of compartments=U+B

P=Probability that an unbound compartment will be occupied at any moment in time.

Mean intensity at the cross section will be u=U P+B=U P+(N−U)=N−U(1−P).

$$Hence N - u = U ( 1 - P ) or U = \frac{( N - u )}{( 1 - P )}$$

Variance of intensity is σ²=U P (1−P)=P U (1−P) based on the binomial distribution.

Substituting: N−u=σ²/P or P=σ²/(N−u).

N can be estimated as the maximum intensity over time. From which P can be computed, from which U and B can be computed using the equations above.

In practice, while somewhat instructive, binding dynamics models such as shown above, as well as random walk models, may not prove as effective to model flow in actual perfused tissue at the level required for medical diagnosis. It is recognized that blood flow patterns can fold back upon themselves, creating time delays in the arrival time intensity ramps, flow rates begin to vary as contrast agent particles become immobilized, and various other factors that are not modeled above can come into play.

In the illustrative embodiments is notable that, despite the limitations of flow-specific binding models, since there tend to be multiple binding sites present within each pixel, with multiple probabilistic events contributing to the intensity, such that the aggregate flows and binding patterns can be effectively approximated using the mean-subtraction approach outlined above. This is because a combination of random variables tends toward the normal distribution (due to the central limit theorem). Thus, mean-subtraction, ideally using a windowed approach, permits computation estimates based on aggregate dynamics that may combine multiple probabilistic effects resulting from blood flow rate changes, accumulation due to binding, changes in concentration, etc. This combination of windowing with aggregate normal distribution motivated dynamics is highly effective in accordance with an illustrative embodiment.

(v.) Optimization Across Measurement Window Boundaries

Once an initial estimate of the intensity due to bound contrast agent has been produced within each window, the estimate can be further refined by analyzing concentrations across multiple measurement windows. For example, in the case of monitoring initial accumulation of contrast agent during the first minute after arrival of the contrast agent bolus at the site of interest, it is expected that the intensity of the bound contrast agent signal is initially fairly small (since no contrast agent was available for binding), and increases over time to the intensity measured in the last sample. Application of filtering based on this expectation can further refine the signal estimates. For example, for contrast agent first arriving at the imaging site in the first measurement window (starting at time t=0), producing an estimated bound contrast agent signal intensity at a given pixel/voxel of I₀, with imaging continuing at one frame per second until the window starting at time point (e.g.) t=50 seconds produces an estimated bound contrast agent signal intensity of I₅₀, the procedure can require that I₅₀>I₀+τ where τ is a constant reflecting the minimum amount of binding that must occur for a pixel to be considered as having a valid signal. Pixels/voxels that do not achieve at least that minimum amount of change in estimated amount of binding can be rejected as not having significant accumulation. It is contemplated that the following various constraints on accumulation rates and amounts can be advantageous in improving the quality of both quantitative and qualitative estimation of the intensity due to bound contrast agent:

(a) Imposition of a maximum intensity constraint at window 0. In other words, if the intensity estimate in window 0 is not low, there should be residual background signal or some other noise source present at the site; i.e. to be considered a valid accumulation site, the approach requires that I₀<minThreshold.

(b) Imposition of a maximum slope constraint. For example, (Iᵢ₊₁−Iᵢ)<maxThreshold; i.e. if accumulation happens too quickly, it is likely due to reasons other than molecularly targeted adherence of contrast particles.

(c) Imposition of the final intensity as a maximum intensity constraint across all samples. In situations where contrast agent has extremely high flow during a few seconds of the bolus arrival, within-window estimates of bound contrast agent accumulation (for example, using the minimum-intensity projection approach described above) can be temporarily inflated due to the presence of flowing contrast particles in many or all acquired samples. As the extremely high flow of contrast agent particles subsides, the intensity decreases to more accurately reflect the actual concentration of bound contrast agent. Using the final estimate of intensity due to bound contrast agent accumulation, I_{final}, as a limit to be applied to all other measurements can be advantageous. For example, in an example where I₂₂>I₅₀, the approach defines an enhanced intensity estimate E such that Eᵢ=min(Iᵢ, I_{final}). Hence, in this example E₂₂=I₅₀, rather than E₂₂=I₂₂.

(d) Enhancement of intensity estimates by making adjustments such that the intensity estimates are uniformly increasing. A straightforward adjustment is made to reduce any intensity level Iᵢ that is greater than any subsequent window's intensity estimate Iⱼ. In other words, for an examination that acquires N measurement windows after contrast agent arrival, the approach calculates an enhanced intensity estimate Eᵢ, such that Eᵢ=min(Iᵢ, Iⱼ) for all j in the interval [i+1, n−1]. As an alternative to use of a maximum value threshold, as is implemented by the min function above, data smoothing techniques can be applied to produce a set of estimates of bound contrast agent intensity that increase monotonically over time.

(e) In various embodiments, the data smoothing can be informed by knowledge of the binding rate properties of the contrast agent. The binding rate properties can be known in advance, or can be inferred through comparison of the dynamic intensity properties occurring at multiple locations in the image.

(f) Also in various embodiments, the data smoothing can be informed by the behavior of neighboring pixels. For example, basing the enhanced signal estimates on the minimum value of the intensities estimated for each window across a group of multiple pixels. Those skilled in the art can recognize that this technique is somewhat akin to Gaussian smoothing for noise reduction, but rather than performing a Gaussian operator, which tends to have an averaging effect, this technique instead employs a spatial minimum filter that takes the weakest signal within a given spatial analysis region. This spatially-derived minimum can be combined across windows to produce an overall minimum as well. This serves to both spatially and temporally reduce the impact of very high flows of contrast agent, but at the cost of some spatial resolution, since multiple pixels are being aggregated to form the spatial minimum.

(g) Constraints on maximum change rates relative to the mean and/or standard deviation. For example, in the example of a piece of tissue-leakage background signal that is entering and exiting a region of interest due to patient or probe motion, there will be a sudden discontinuity in the intensity and the standard deviation. This discontinuity can be detected and used to reject either a single window's estimate for that pixel/voxel, or in the example of conservative imaging protocols, reject the entire sequence of window estimates for that pixel/voxel.

(vi) Confidence Tracking

As described above, the applied cross-window constraints can be used to produce enhanced estimates of bound contrast agent intensity for each pixel/voxel of each measurement window, and can also be used to track the validity of the signals associated with specific pixels/voxels within a single window or across multiple windows. Advantageously, a data structure reflecting pixel/voxel location and measurement window times indicating valid, enhanced, or invalid signal can be maintained. This data structure can be provided as a simple bitmap image indicating signal validity across all time, or can be a more detailed representation indicating specific time intervals and causes of invalidation or enhancement. Tracking the cause for invalidation (for example, insufficient change over time, or transformation of background behavior between occlusatory and additive) can be advantageous in explaining results to end users of the system, and to assigning probabilities (certainty levels, etc.) to the resulting bound contrast agent accumulation estimates. Similarly, tracking types of enhancement (if any) that have been applied to each region, such as enhancement based on B-mode intensity to overcome shadow artifacts, or enhancement based on accumulation detected after background occlusion was initiated, can be very helpful for use by later analysis stages (by human radiologist or a computer program) to gauge confidence level in the result.

D. Region of Interest (ROI) Segmentation

The procedure 200 receives estimates of contrast agent (bound, unbound and/or background) 252 from step 250 above, and now performs step 260.

(i) Automatic Delineation of Regions of Interest (ROIs)

Suspected tumor regions can be automatically detected, and can also be depicted graphically, based on spatial analysis of the estimated bound intensities present in one or more measurement windows. For each measurement window, a synthetic image, known as the residual image, is formed from the best estimates of bound contrast agent present at each location. Ideally background has been removed from this residual image, for example using the subtraction techniques or statistical techniques described variously in section D above. Once the residual image has been formed, it can be processed spatially to further remove noise and increase spatial signal continuity. In an embodiment this noise-reduction and spatial-signal enhancement is achieved via (e.g.) a grayscale morphological closing. The result of the closing is then segmented, dividing it into regions in which significant bound contrast agent is present and regions where it is not present. One way to achieve this segmentation is to construct a threshold map from the enhanced residual image. If the estimated bound contrast intensity exceeds a threshold (T), then the map is set to 1 (i.e. it contains significant amounts of bound contrast agent) for that location. If the signal falls below the threshold, then the map is set to 0 (i.e. it contains no significant amounts of bound contrast agent) for that location.

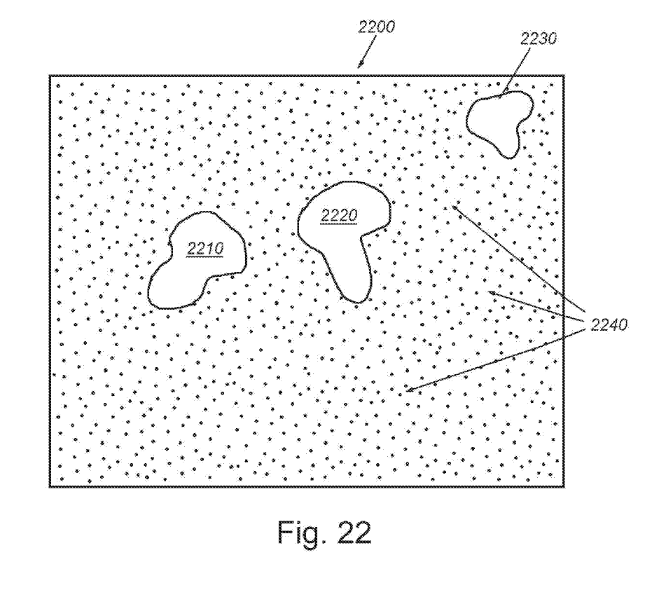

The segmentation threshold (T) is computed based on spatial statistical analysis of the residual image. This can be computed on a per-window basis, or across all measurement windows. For the embodiment described here, the spatial statistics are computed on a per-window basis. First, the overall spatial mean (across all pixels) and standard deviation (across all pixels) of each residual image is computed. The threshold (T) is determined by computing the number (k) of spatial standard deviations (σₛ) above the spatial mean (μₛ). That is T=μₛ+kσₛ. A high k value will result in fewer detections but fewer false positives as well. It is contemplated that for use in imaging disease, for example, cancer tissue using ultrasound, k can be set equal to approximately 3, excluding all but the strongest signals. The result is a segmented image 2200 associated with each measurement window, as shown in FIG. 22. This exemplary segmented image representation shows regions where the estimated bound contrast agent accumulation exceeds a threshold T computed based on an estimated bound contrast agent intensity value at least k=3 standard deviations above the mean intensity of the image. These regions 2210, 2220 and 2230 are shown as white patches surrounded by a substantially black field (represented herein with dot shading) 2240.

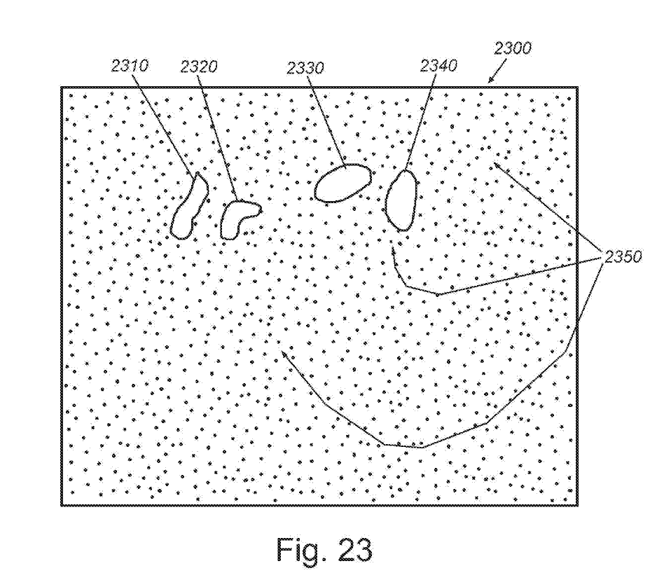

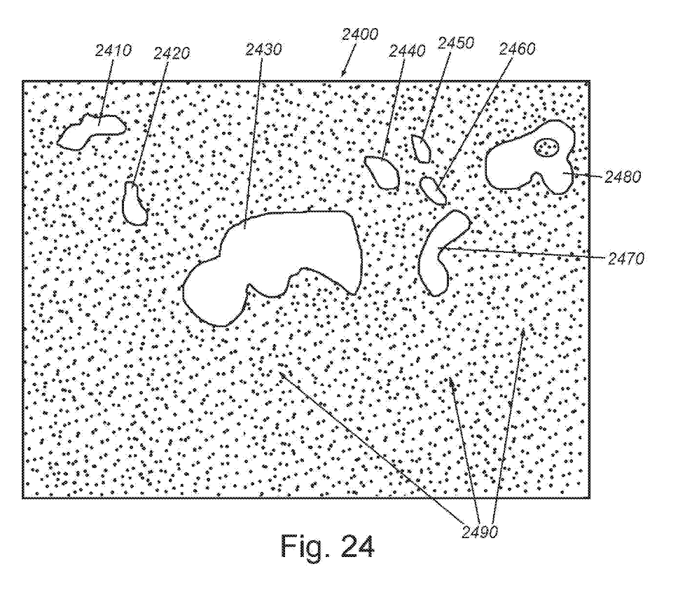

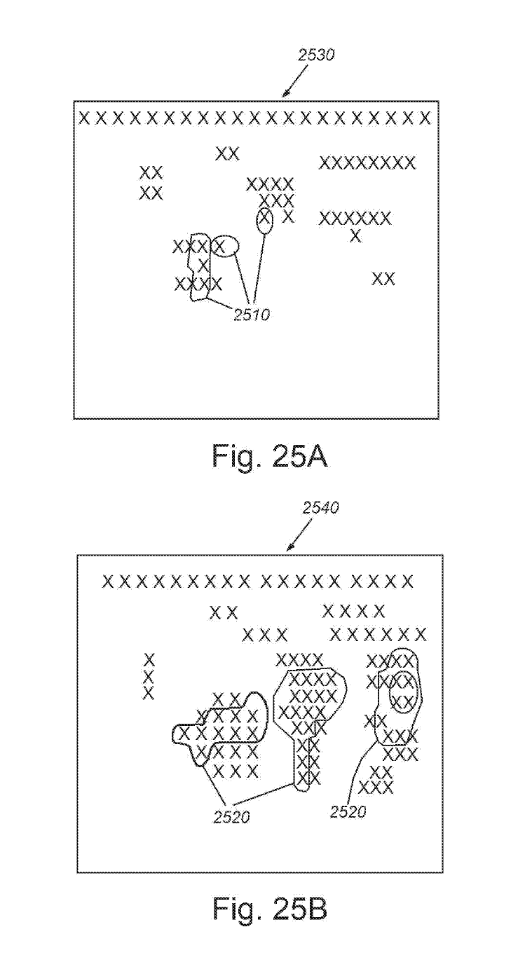

When using an approach such as the minimum intensity projection described above, at the earliest instant after contrast agent arrival, there will be no detections since it takes a certain amount of elapsed time (corresponding to the measurement window width) to detect the accumulation of the targeted signals. For a window width of (e.g.) W=15, using the minimum intensity projection approach the first signals occur W samples (e.g. 15 seconds at a sample rate of 1 Hz) after the arrival of the contrast agent. As represented in the exemplary segmented image 2300 of FIG. 23, only a few, relatively small regions 2310, 2320, 2330 and 2340 (shown as white patches surrounded by a (dot-shaded) dark field 2350) are detected during this initial arrival phase. As time progresses, images derived from later measurement windows show increased accumulation of bound contrast agent. The representative segmented image 2400 in FIG. 24 shows the detection results at the last instance, for a measurement window of (e.g.) size 15 that ends (e.g.) 35 seconds after the initial arrival of contrast agent. In this image 2400, larger/more-numerous, exemplary regions 2410, 2420, 2430, 2440, 2450, 2460, 2470 and 2480 (white patches) surrounded by a substantially black (dot-shaded) field 2490.

Since the arrival time of the contrast agent, the duration of imaging, and technical acquisition parameters, such as sample rate can vary with each patient and potentially with each practitioner and/or model of imaging equipment involved in the imaging procedure, the number of images available for processing can also vary. Thus, preselecting any one instance in time may not be ideal to detect the targeted signals. Instead, an embodiment of this approach advantageously employs multiple segmented images, or even the entire sequence of segmented images, to determine the final detected regions. In an embodiment, a final segmented image is computed by taking the maximum at each pixel using (e.g.) across all of the segmented images. In an embodiment, after the final segmented image is computed, the outlier regions are removed to filter out regions with anomalous region properties. By way of example, the final decision as to whether a threshold-exceeding detection occurred is determined at a time T_{final} (e.g. 35 seconds) after contrast agent arrival. However, in this approach the final detected image can be sensitive to measurement noise at each pixel in all frames, i.e. a single intensity burst at any moment in time can impact the final image. In an alternate embodiment, when performing the thresholding computation, the value in the last image is used as a limit on the intensities of all other frames. In other words, since contrast agent is known to accumulate over time, any intensity that exceeds that of the final intensity is rejected as an outlier. In this way, only noise in the last image will impact the final intensity. Various other embodiments are contemplated, such as imposing ranges on the permissible values of the slope of the image intensity over time.

To further mitigate the impact of measurement noise, such as that introduced by high concentrations of flowing contrast agent particles, it can be desirable to apply additional cross-window optimization to the binary detection images. For example, it is contemplated to eliminate outliers by eliminating from the detected regions any pixel that is not present in the final detected region, i.e. to be considered a valid signal for a window that ends 20 seconds after contrast agent arrival, the signal should also be present in the window that ends 38 seconds after contrast agent arrival. This eliminates evanescent signals that only appear for a short number of measurement windows and then dissipate. However, this condition can create sensitivity to drop-out noise in the last measurement window. Alternatively, the procedure can require that signals persist for a particular time duration, or in a certain number of measurement windows, in order to be considered.

(ii) Region of Interest Delineation

After the final segmented image has been computed, region of interest outlines can be generated to delineate the targeted signals-of-interest. A binary image closing is performed on the segmented image. Image closing is performed by applying an image dilation followed by an image erosion. A local kernel is used to specify the amount of closing, that is, the number of pixels to close. In an exemplary embodiment a disk-shaped kernel with a size of (e.g.) 5 pixels can be used. Other shapes can be employed in a manner clear to those of skill. By way of example, the output segmented closed image can then be processed by the binary Canny edge detector as described in A Computational Approach To Edge Detection, by J. Canny, IEEE Trans. Pattern Analysis and Machine Intelligence, 8(6):679-698, 1986.

The output of this edge detection is an image containing only binary outlines around the targeted signals. This can take the form as shown by the schematic image representations of FIGS. 25A and 25B, which respectively show the detection outlines 2510 and 2520 overlaid on top of the raw image 2530 and 2540 associated with the last frame of the measurement window. At the initial stage of detection shown in FIG. 25A, (e.g.) 15 seconds after the contrast agent arrival, there are fewer regions detected as evident by the lack of bright signals (cross hatching) in the image. However, (e.g.) four (4) seconds later, the image shows a significant increase in signal strength across the entire image (larger/additional cross-hatched regions). As a result, the number and size of the detected regions also increase as shown in the image 2540 of FIG. 25B. Note that the particular schematic diagram contemplates an exemplary measurement window width of 15 using the minimization approach for detection. However, the statistical approach and/or a differing measurement window size can be employed in alternate embodiments.

E. Presentation of Analysis Results to End Users

The procedure 200 receives image(s) with the regions delineated 262 from step 260 above, and now performs step 270. In general, presentation of results to end users can include providing, on a GUI and/or via a printout or stored data a graphical image with enhancements and color coding that accentuates the tumor region and otherwise removes undesired background. This presentation of data assists the user—typically a medical practitioner—in determining the nature and extent of tumorous growth in the tissue, which can guide subsequent treatment options for the patient.

III. Image-Guided Procedures

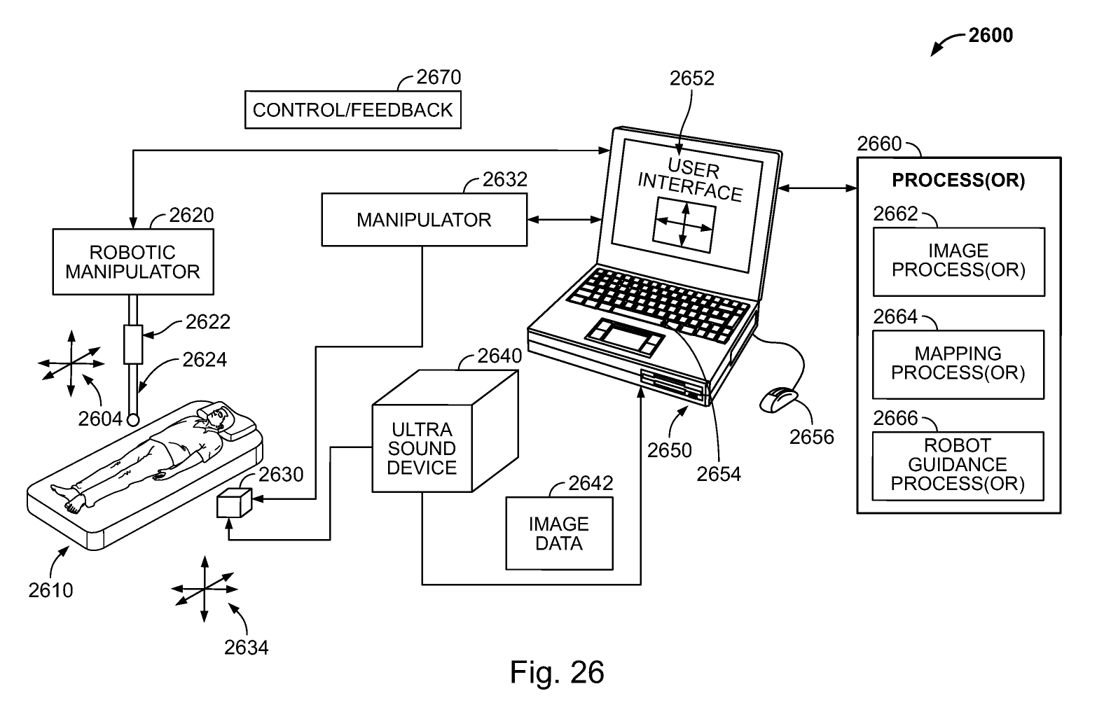

FIG. 26 shows a generalized diagram of an arrangement 2600 for performing an invasive medical procedure using imagery generated (e.g. in real-time or near-real-time) according to the various systems and methods described and/or contemplated herein. The procedure, performed on the internal regions of an exemplary patient 2610 can be any form of invasive technique—including, but not limited to, biopsies, laparoscopies, insertion of implanted devices, targeted radiation therapy (including proton beam, radiotherapy beam, microwave ablation treatment, etc.), surgeries (using various instruments, such as forceps, probes, scalpels, lasers and/or electo-cauterizers). The depicted arrangement 2600 shows a robotic manipulator (e.g. a multi-axis arm, or other device) 2620 directing a surgical instrument 2622, having an instrument tip (e.g. a surgical instrument as described above, biopsy needle, etc.) 2624, into an internal region of the patient 2610. The patient's internal region can be monitored using (e.g.) an ultrasound probe 2630 that is interconnected to a corresponding ultrasound device 2640, and senses in B-mode and/or Contrast-mode. In various embodiments, the patient's internal region can be monitored using Doppler-mode ultrasound, Magnetic Resonance Imaging (MRI), CT scan, fluoroscopy, a surgical navigation and tracking system, and/or various other means for tracking and/or imaging internal portions of the patient's body. The probe 2630 is handled with respect to the patient 2630 using an appropriate manipulator 2632, which can also be a robotic arm or a fixed mechanism (for example, a mount that maintains the probe in a stationary location with respect to the patient's body. It is contemplated that one or both of the manipulators 2620 and 2632 can be robotic or can include the hand of a practitioner. Hence, the instrument 2622 and/or probe 2632 of the arrangement 2600 can be guided manually in some embodiments. Where manual guidance is part of the invasive procedure, the term “manipulator” as used herein should be taken broadly to include a human-guided device (e.g. a semi-automated surgical device) and/or the practitioner's hand(s).

In an automated arrangement, the instrument's manipulator 2620 defines a 2D or 3D coordinate space 2624 that can include both translation and rotation—depending upon the degrees of freedom available for manipulator motion. Likewise, the probe 2630 can define a 2D or 3D coordinate space 2634 which is associated with image data 2642 transmitted from the ultrasound device 2640.

The transmitted image data 2642 is received by an appropriate interface in a computing device—for example, a PC, tablet, handheld computer (e.g. smartphone), laptop, server or cloud-computing environment 2650. The computing device 2650 includes an appropriate graphical user interface (GUI), having (e.g.) display/touchscreen 2652, keyboard 2654, mouse 2656, etc. The computing device includes a process(or) 2660 with appropriate operating system. The process(or) 2660 drive an image processing module 2662 that performs the various systems and methods for handing image data as described herein. The results of such processing can be displayed on the interface display 2652 and/or other associated display screens—including virtual reality (VR) or augmented reality (AR) headset(s) worn by clinicians or others. The image data is processed so as to render features of interest within the scanned internal region clear and more-defined. This assists both automated and manually guided invasive procedures in properly navigating an inserted instrument tip 2624.

The process(or) 2660 also includes a coordinate space mapping process/module 2664 that translates the image coordinate space 2634 into the instrument manipulator's coordinate space 2624 (and vice versa). The translation can be based upon an appropriate kernel that is established during an initial calibration or training process. Such procedures for calibration/training are understood by those of skill. The mapped coordinates can be provided to a robot guidance process/module 2666. The guidance process/module 2666 receives the image data and, based on user inputs (via interface 2652) and/or other automated commands controls guidance the surgical instrument tip and (optionally) receives feedback (block 2670) from the tip and/or manipulator 2620. Guidance can enable the tip 2624 to drive into a region of interest to collect a tissue sample (a biopsy), and withdraw therefrom. Alternatively, guidance can enable the tip 2624 to navigate around the (now-defined) edges of a region to surgically excise it or perform other appropriate surgical tasks.

The system can establish and store relationships in 3D space between regions of interest and/or biological margins identified during pre-surgical or intraoperative contrast-mode imaging and landmarks within the patient's body that can be identified through B-mode ultrasound, contrast-mode ultrasound, Doppler mode ultrasound, MRI, CT scan, fluoroscopy, and/or various other means of imaging. In various embodiments, landmarks can include fiducial markers attached to tissues that can be recognized through non-contrast-mode imaging. Fiducial markers can be attached physical features, such as a surgical staple or clamp, may be features induced on the tissue itself, such as a laser-inscribed surgical tattoo, or may be features that are a natural part of the tissue, such as an easily-observed anatomical feature, selected by the user (surgeon), or by the automated system for use as a motion-tracking marker.

After the contrast agent is no longer present in sufficient quantity for useful contrast-mode imaging, the system can remember where the biological margins and/or regions of interest that were identified through contrast mode ultrasound were located relative to the landmarks that can be identified without contrast agent imaging. The system can thereby guide various surgical tools relative to the biological margins and/or regions of interest although these margins and/or regions may not remain identifiable through contrast mode imaging during surgery. Information about the location of the margins and/or regions can be added to images that can be taken during surgery, so that the surgeon can know the target location(s) during surgery. Operative images taken during surgery can include images taken with B-mode ultrasound, Doppler-mode ultrasound, MRI, CT scan, fluoroscopy, or others, and information about the locations of regions and/or margins identified with contrast-mode ultrasound can be added to the operative images. Landmarks that are available in the operative images can be used to anchor the locations of the margins and/or regions added to the operative images. The system can fuse information obtained in contrast-mode ultrasound with information about the absolute location of tissues obtained through recognition of landmarks in non-contrast mode imaging. In various embodiments, coordinate space mapping can also include surgical tracking and surgical navigation system location mapping, as described in U.S. patent application Ser. No. 15/909,282, the entire disclosure of which is incorporated herein by reference. The system can create a surgical tracking and surgical navigation system map by creating relationships in 3D space between the margins and/or regions identified using contrast mode and landmarks identifiable during operative imaging.

In various embodiments, a location map can incorporate data representing the confidence with which a feature is known to be present. This confidence data can be presented as gradients and can be color-coded within an image. Confidence data can be presented as a simple yes/no indicating whether or not the confidence level is above a predetermined threshold. Regions with a confidence level above the predetermined threshold can be indicated showing that sufficient confidence exists in those regions for surgery to proceed with confidence.

In various embodiments, the system can also construct location maps that include highly perfused tissue identified using contrast mode ultrasound. Blood flow and tissue perfusion can be identified by administering a non-molecularly targeted ultrasound contrast agent, and tissue perfusion information can be obtained indicating areas of blood supply flow. The tissue perfusion information can be obtained by analyzing flow patterns of the non-molecularly targeted ultrasound contrast agent. Blood flow and tissue perfusion can be identified by administering a molecularly targeted ultrasound contrast agent, and tissue perfusion information can be obtained indicating areas of blood supply flow. The tissue perfusion information can be obtained by performing statistical analysis to analyze flow patterns of the molecularly targeted ultrasound contrast agent. This statistical analysis can include performing a maximum intensity projection to approximate blood flow patterns by highlighting areas with large amounts of contrast agent. In various embodiments, a percentage intensity projection can be used to highlight areas with large amounts of contrast agent flow by using a percentage intensity in a range of approximately 70% to approximately 90%. In various embodiments, a percentage intensity projection can be used to highlight areas with large amounts of contrast agent flow by using a percentage intensity of approximately 80%. In various embodiments, the statistical analysis can include subtracting a, estimate of bound contrast agent from the maximum intensity projection, resulting in an estimate of flowing contrast agent. The estimate of bound contrast agent can be a minimum intensity projection in a range of approximately 1% to approximately 10%. The estimate of bound contrast agent can be a minimum intensity projection of approximately 5%. Various other statistical means can be used to determine the estimate of bound contrast agent, as explained above, and the estimate of bound contrast agent can be subtracted from various estimates of flowing contrast agent to increase the accuracy of the estimates of flowing contrast agents.

The system can identify regions of highly perfused tissue that can include blood vessels using contrast mode ultrasound, and can remember the locations of these highly perfused tissues relative to one or more landmarks. Fluid flow in arteries can be detected via (e.g.) Doppler ultrasound using an ultrasonic transducer mounted on the surgical probe, or using targeted or non-targeted contrast agent in contrast-mode ultrasound. The system can use this information to create maps of vasculature locations.

In various embodiments, the surgical navigation system location mapping system described herein can be used to map and track highly-perfuse tissue that can include blood vessels, in addition to the above described computationally-enhanced molecularly targeted ultrasound findings. The surgical navigation system location mapping described above can be used in conjunction with various other imaging modalities described herein, and features described above can be combined with or added to any of the various other imaging modalities described herein.

Information about, or images of, highly perfused tissue that has been found using Doppler or contrast-enhanced ultrasound or other types of angiography can be composited with information about, or images of, locations of molecularly-targeted contrast agent. Some highly perfused tissue is cancerous while others are simply highly-perfused tissue, and the inclusion of information about bound molecularly-targeted contrast agent can help to indicate which highly-perfused tissue is cancerous and which is not. The molecularly bound contrast agent indicates which tissues are cancerous, and the perfusion data can indicate how much cancerous tissue exists and where the boundaries or margins of the cancerous tissue are located. A user can use the composited information to remove all of the tumor region, and to ensure the blood supply has been terminated to the tumor regions. In various embodiments, the user can use the composited information to target regions of interest using, for example, a biopsy tool while simultaneously avoiding contact with blood vessels. The system can establish and store relationships in 3D space between these highly perfuse tissues and landmarks so that they can be targeted or avoided during surgery.

As described above, in addition to autonomous guidance, the guidance procedure can be manually or semi-automatically (e.g. a surgeon directly operating an automated manipulator) based on visual observation of a display and visual feedback as the tip moves within the region. The use of enhanced imagery as described herein increases the accuracy and effectiveness of such manual procedures.

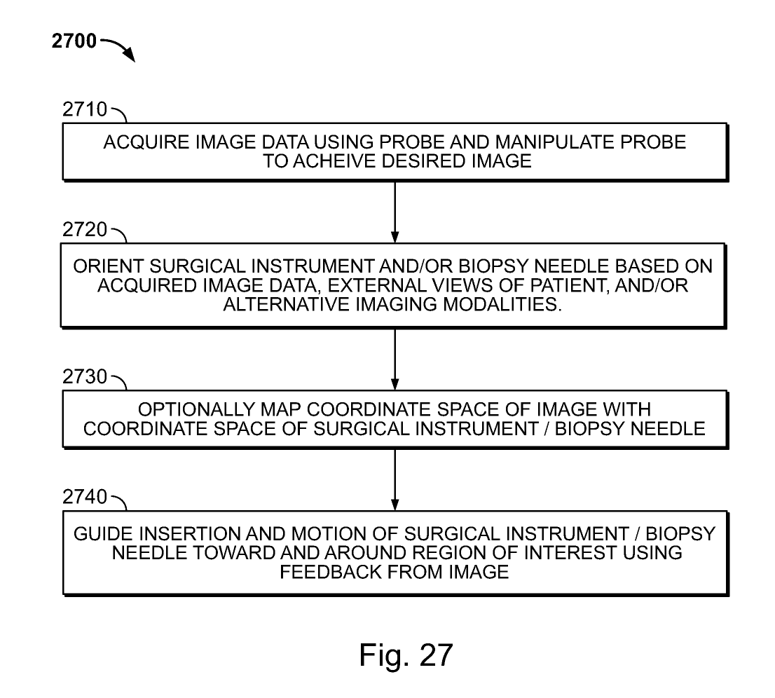

Briefly, a process 2700 for image guided invasive procedures is shown in the flow diagram of FIG. 27. The procedure 2710 entails applying a probe to the patient's body with respect to a region of interest—i.e. to which a surgical procedure is being performed. The probe can be manipulated via manual or automated techniques to obtain a desired image (step 2710). A surgical instrument, biopsy needle or other invasive tip can be oriented with respect to the imaged region of interest, external views of the patient, and/or alternative imaging modalities (B-mode, MRI, CT scan, fluoroscopy, etc.) in a manner that allows it to invade the area of treatment in a manner that allows the tip to be imaged by the probe and the associated imaging device (step 2720). The coordinate space of the acquired images can be (optionally) mapped with respect to the coordinate space of the surgical instrument or needle tip. In various embodiments, the acquired images can be composited with additional operative images obtained in real-time or near real-time, or can include information that was previously gathered, including, by way of non-limiting examples, regions of interest located previously using molecularly-targeted contrast agent and contrast mode ultrasound, information on the locations of blood vessels that can include flow information, information on the locations of nerves, and/or other information or images. In an automated environment this entails training and calibration parameters/kernel(s). In a manual environment, the clinician's eyes and brain generally provide the necessary mapping (step 2730). Then, in step 2740, the image data/display is used to provide feedback in guiding the tip toward and within/around the region of interest in a manner clear to those of skill.

IV. Conclusion

It should be clear that the system and method described above effectively addresses disadvantages encountered when performing contrast-based ultrasound imaging in the presence of bound contrast agent, such as microbubbles. The system and method operates in a manner that can be non-destructive to both microbubbles and surrounding tissue being scanned, using conventional device settings in combination with advanced and novel image processing techniques. The techniques can be performed with reasonable processing overhead. In addition to filtering unwanted background information, they also address contrast-agent-generated occlusion of features. The system and method effectively addresses occlusion of tissue (which can contain background signal) by contrast agent in acquired images to generate a more accurate result. These results, which are generated for either a human user to examine, or for an automated diagnosis tool to analyze, are more-reliable, and allow for better diagnostic outcomes. Moreover, the image data provided by the system and method herein allow for image-guided procedures (automated and/or manual) that allow for clearer and more accurate guidance of a surgical instrument tip, biopsy needle, etc., with respect to an otherwise obscured or occluded region of interest.

The foregoing has been a detailed description of illustrative embodiments of the invention. Various modifications and additions can be made without departing from the spirit and scope of this invention. Features of each of the various embodiments described above may be combined with features of other described embodiments as appropriate in order to provide a multiplicity of feature combinations in associated new embodiments. Furthermore, while the foregoing describes a number of separate embodiments of the apparatus and method of the present invention, what has been described herein is merely illustrative of the application of the principles of the present invention. For example, as used herein various directional and orientational terms (and grammatical variations thereof) such as “vertical”, “horizontal”, “up”, “down”, “bottom”, “top”, “side”, “front”, “rear”, “left”, “right”, “forward”, “rearward”, and the like, are used only as relative conventions and not as absolute orientations with respect to a fixed coordinate system, such as the acting direction of gravity. Moreover, a depicted process or processor can be combined with other processes and/or processors or divided into various sub-processes or processors. Such sub-processes and/or sub-processors can be variously combined according to embodiments herein. Likewise, it is expressly contemplated that any function, process and/or processor herein can be implemented using electronic hardware, software consisting of a non-transitory computer-readable medium of program instructions, or a combination of hardware and software. Accordingly, this description is meant to be taken only by way of example, and not to otherwise limit the scope of this invention.

What is claimed is:

1. A method for performing a surgical procedure comprising the steps of:
administering an ultrasound contrast agent to a patient;
performing ultrasound imaging of at least a portion of the patient using contrast-mode ultrasound to obtain at least one ultrasound image of the patient;
performing statistical analysis of the at least one contrast-mode ultrasound image to enhance the clarity of the at least one contrast-mode ultrasound image; and
guiding a surgical tool using information gained from the at least one enhanced contrast mode ultrasound image.

2. The method of claim 1, wherein performing statistical analysis of the at least one ultrasound image further comprises performing statistical analysis of multiple-image windows to identify pixel intensity caused by molecularly bound contrast agent.

3. The method of claim 1 wherein performing ultrasound imaging of a portion of the patient using contrast-mode ultrasound to obtain at least one ultrasound image of the patient further comprises performing operative imaging of the patient to obtain operative images of the patient using contrast-mode ultrasound, and wherein guiding a surgical tool using information gained from the at least one enhanced contrast mode ultrasound image further comprises guiding the surgical tool using the enhanced operative images.

4. The method of claim 1 further comprising performing operative imaging of the patient to obtain operative images using B-mode ultrasound, Doppler-mode ultrasound, Magnetic Resonance Imaging (MRI), CT scan, or fluoroscopy, and wherein guiding a surgical tool using information gained from the at least one enhanced contrast mode ultrasound image further comprises adding the information gained from the contrast-mode ultrasound to the operative images.

5. The method of claim 4, wherein adding the information gained from the contrast-mode ultrasound to the operative imaging further comprises annotating the operative images to include regions of interest determined from the statistical analysis of pixel intensity in the contrast-mode ultrasound.

6. The method of claim 5, further comprising tracking features using B-mode feature tracking relative to key landmarks, and updating locations of regions of interest relative to tracked features.

7. The method of claim 4, wherein adding the information gained from the contrast-mode ultrasound to the operative imaging further comprises annotating the operative images to include biological margins determined from the statistical analysis of pixel intensity in the contrast-mode ultrasound.

8. The method of claim 1 further comprising performing operative imaging of the patient to obtain operative images, and wherein guiding a surgical tool using information gained from the at least one enhanced contrast mode ultrasound image further comprises annotating the operative images to include regions of interest determined from the statistical analysis of pixel intensity in the contrast-mode ultrasound, the annotating relative to a landmark.

9. The method of claim 1 further comprising performing operative imaging of the patient to obtain operative images, and wherein guiding a surgical tool using information gained from the at least one enhanced contrast mode ultrasound image further comprises annotating the operative images to include biological margins determined from the statistical analysis of pixel intensity in the contrast-mode ultrasound, the annotating relative to a landmark.

10. The method of claim 1, further comprising adding a color-coding to the one or more ultrasound images to indicate confidence levels in different portions of the one or more ultrasound images.

11. The method of claim 4, further comprising adding a color-coding to the one or more ultrasound images to indicate confidence levels in different portions of the one or more ultrasound images, the color-coding adaptive to track the motion or imaging perspective of the B-mode image.

12. The method of claim 1, further comprising indicating to a user that a predetermined confidence requirement for confidence in the enhanced ultrasound image has been met so that surgery can proceed with confidence.

13. A method for performing a surgical procedure comprising the steps of:
administering at least one ultrasound contrast agent to a patient;
performing at least one ultrasound imaging of at least a portion of the patient using contrast-mode ultrasound to obtain at least one ultrasound image of the patient;
measuring tissue perfusion in at least one of the at least one ultrasound images of the patient to obtain tissue perfusion information indicating areas of blood supply flow;
identifying locations of bound contrast agent in at least one of the at least one ultrasound images of the patient to obtain bound contrast location information;
integrating the tissue perfusion information and the bound contrast location information into a correlated information set that describes perfusion information and bound contrast location information simultaneously; and
guiding a surgical tool using the correlated information set.

14. The method of claim 13, wherein integrating the tissue perfusion information and the bound contrast location information into a correlated information set further comprises integrating the tissue perfusion information and the bound contrast location information into an integrated image.

15. The method of claim 13, wherein administering at least one ultrasound contrast agent to a patient further comprises administering a molecularly targeted ultrasound agent, and wherein identifying locations of bound contrast agent in at least one of the at least one ultrasound images of the patent further comprises annotating a region of interest indicated by molecularly bound contrast agent.

16. The method of claim 15, wherein guiding a surgical tool using the correlated information set further comprises guiding a surgical instrument to the region of interest, and further comprises avoiding areas of blood supply flow.

17. The method of claim 15, wherein guiding a surgical tool using the correlated information set further comprises guiding a surgical instrument to remove the region of interest, and further comprises disrupting blood supply flow to the region of interest.

18. The method of claim 15, wherein administering at least one ultrasound contrast agent to a patient further comprises administering a non-molecularly targeted ultrasound contrast agent, and wherein measuring tissue perfusion in at least one of the at least one ultrasound images of the patient to obtain tissue perfusion information indicating areas of blood supply flow further comprises analyzing flow patterns of the non-molecularly targeted ultrasound contrast agent.

19. The method of claim 15, wherein measuring tissue perfusion in at least one of the at least one ultrasound images of the patient to obtain tissue perfusion information indicating areas of blood supply flow further comprises performing statistical analysis to analyze flow patterns of the molecularly targeted ultrasound contrast agent.

20. The method of claim 19, wherein performing statistical analysis further comprises performing a maximum intensity projection to approximate blood flow patterns.

21. The method of claim 20 wherein performing statistical analysis further comprises determining a difference between the maximum intensity projection and a bound contrast agent projection, wherein the bound contrast agent projection is acquired via minimum intensity projection or statistical estimation.

22. The method of claim 19, wherein annotating a region of interest indicated by molecularly bound contrast agent further comprises defining a biological margin using statistical analysis.

23. The method of claim 19, wherein performing statistical analysis further comprises performing a percentage intensity projection.

24. The method of claim 23, wherein performing a percentage intensity projection further comprises performing a percentage intensity projection in a range of approximately 70% to approximately 90%.

25. The method of claim 23, further comprising subtracting a minimum intensity projection from the percentage intensity projection, the minimum intensity projection in a range of approximately 1% to approximately 10%

26. A system for performing a surgical procedure in which at least one ultrasound contrast agent is administered to a patient comprising:
at least one ultrasound image of at least a portion of the patient taken using contrast-mode ultrasound to obtain the at least one ultrasound image;
a measuring process that measures tissue perfusion in at least one of the at least one ultrasound images of the patient to obtain tissue perfusion information indicating areas of blood supply flow;
and identification process that identifies locations of bound contrast agent in at least one of the at least one ultrasound images of the patient to obtain bound contrast location information;
an integration process that integrates the tissue perfusion information and the bound contrast location information into a correlated information set that describes perfusion information and bound contrast location information simultaneously; and
a guidance process that physically guides a surgical tool using the correlated information set.
